(19) United States
(12) Patent Application Publication
(10) Pub. No.: US 2007/0244173 A1
(43) Pub. Date: Oct. 18, 2007

(54) 6,7-Dihydroxy-8-Phenyl-3,6,7,8-Tetrahydro-Chromeno [7,8-d] Imidazole Derivatives and Their Use as Gastric Acid Secretion Inhibitors

(75) Inventor: Wilm Buhr, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/578,801

(22) PCT Filed: Oct. 18, 2006
(86) PCT No.: PCT/EP05/51822

§ 371 Date: Oct. 18, 2006

(30) Foreign Application Priority Data
Apr. 26, 2004 (EP) 04009793.3

(51) Int. Cl. A61K 31/4188; C07D 491/04; C07D 491/14

(52) U.S. Cl. 514/394; 548/300.4; 548/302.1

(57) ABSTRACT

The invention provides compounds of the formula 1, 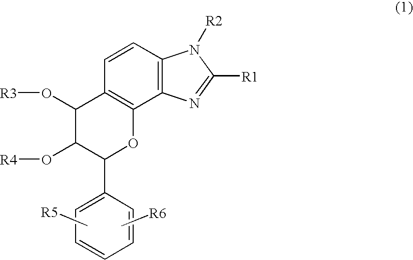 in which the substituents and symbols are as defined in the description. The compounds inhibit the secretion of gastric acid.

### TECHNICAL FIELD

The invention relates to novel compounds which are used in the pharmaceutical industry as active compounds for the production of medicaments.

### BACKGROUND ART

In the international patent application WO 97/47603 (which corresponds to the U.S. Pat. No. 6,465,505), benzimidazole derivatives having a very specific substitution pattern are disclosed, which are said to be suitable for inhibition of gastric acid secretion and thus can be used in the prevention and treatment of gastrointestinal inflammatory diseases.

In the European patent application EP 0266326 (which corresponds to U.S. Pat. No. 5,106,862) benzimidazole derivatives having a very broad variety of substituents are disclosed, which are said to be active as anti-ulcer agents.

The International patent applications WO 98/54188 and WO 01/72755 disclose tricyclic imidazopyridine derivatives having a very specific substitution pattern, which compounds are said to be suitable for treating gastrointestinal disorders.

The International patent application WO 04/054984 discloses bicyclic benzimidazole derivatives and the International patent application WO 04/087701 discloses tricyclic benzimidazole derivatives, where in both cases the compounds are likewise said to be suitable for treating gastrointestinal disorders.

### DISCLOSURE OF INVENTION

### Technical Problem

A whole series of compounds are known from the prior art which inhibit gastric acid secretion by blockade of the H+/K+-ATPase. The compounds designated as proton pump inhibitors (PPI's), for example omeprazole, esomeprazole, lansoprazole, pantoprazole or rabeprazole, bind irreversibly to the H+/K+-ATPase. PPI's are available as therapeutics for a long time already. A new class of compounds designated as reversible proton pump inhibitors (rPPI's) or as acid pump antagonists (APA's) bind reversibly to the H+/K+-ATPase. Although rPPI's or APA's are known for more than 20 years and many companies are engaged in their development, no rPPI or APA is at present available for therapy. The technical problem underlying the present invention is therefore to provide acid pump antagonists which can be used in therapy.

### TECHNICAL SOLUTION

The invention relates to compounds of the formula 1 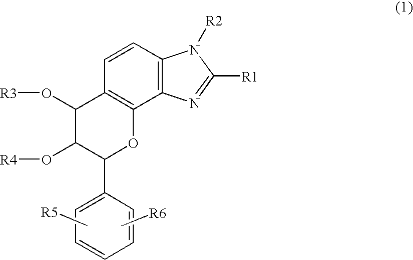
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, halogen, 1-4C-alkyl or fluoro-1-4C-alkyl
R6 is hydrogen, halogen, 1-4C-alkyl or fluoro-1-4C-alkyl
   and the salts of these compounds.

1-4C-Alkyl denotes straight-chain or branched alkyl radicals having 1 to 4 carbon atoms. Examples which may be mentioned are the butyl, isobutyl, sec-butyl, tert-butyl, propyl, isopropyl, ethyl and methyl radicals.

3-7C-Cycloalkyl denotes cyclopropyl, cyclobutyl, cyclopentyl, cyclohexyl and cycloheptyl, among which cyclopropyl, cyclobutyl and cyclopentyl are preferred.

3-7C-Cycloalkyl-1-4C-alkyl denotes one of the abovementioned 1-4C-alkyl radicals which is substituted by one of the abovementioned 3-7C-cycloalkyl radicals. Examples which may be mentioned are the cyclopropylmethyl, the cyclohexylmethyl and the cyclohexylethyl radicals.

1-4C-Alkoxy denotes radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

1-4C-Alkoxy-1-4C-alkyl denotes one of the abovementioned 1-4C-alkyl radicals which is substituted by one of the abovementioned 1-4C-alkoxy radicals. Examples which may be mentioned are the methoxymethyl, the methoxyethyl and the butoxyethyl radicals.

1-4C-Alkoxycarbonyl (—CO-1-4C-alkoxy) denotes a carbonyl group to which is attached one of the abovementioned 1-4C-alkoxy radicals. Examples which may be mentioned are the methoxycarbonyl (CH₃O—C(O)—) and the ethoxycarbonyl (CH₃CH₂O—C(O)—) radicals.

2-4C-Alkenyl denotes straight-chain or branched alkenyl radicals having 2 to 4 carbon atoms. Examples which may be mentioned are the 2-butenyl, 3-butenyl, 1-propenyl and the 2-propenyl (allyl) radicals.

2-4C-Alkynyl denotes straight-chain or branched alkynyl radicals having 2 to 4 carbon atoms. Examples which may be mentioned are the 2-butynyl, the 3-butynyl and, preferably, the 2-propynyl (propargyl radicals).

Fluoro-1-4C-alkyl denotes one of the abovementioned 1-4C-alkyl radicals which is substituted by one or more fluorine atoms. Examples which may be mentioned are the fluoromethyl, the difluoromethyl, the trifluoromethyl, the 2-fluoroethyl, the 2,2-difluoroethyl, the 1,2,2-trifluoroethyl, the 2,2,2-trifluoroethyl, the 1,1,2,2-tetrafluoroethyl or the perfluoroethyl radical.

Fluoro-1-4C-alkoxy-1-4C-alkyl denotes one of the abovementioned 1-4C-alkyl radicals which is substituted by a fluoro-1-4C-alkoxy radical. Here, fluoro-1-4C-alkoxy denotes one of the abovementioned 1-4C-alkoxy radicals which is fully or predominantly substituted by fluorine. Examples of fully or predominantly fluorine-substituted 1-4C-alkoxy which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals. Examples of fluoro-1-4C-alkoxy-1-4C-alkyl radicals which may be mentioned are 1,1,2,2-tetrafluoroethoxymethyl, the 2,2,2-trifluoroethoxymethyl, the trifluoromethoxymethyl, the 1,1,2,2-tetrafluoroethoxyethyl, the 2,2,2-trifluoroethoxyethyl, the trifluoromethoxyethyl and preferably the difluoromethoxymethyl and the difluoromethoxyethyl radical.

Hydroxy-1-4C-alkyl denotes abovementioned 1-4C-alkyl radicals which are substituted by a hydroxyl group. Examples which may be mentioned are the hydroxymethyl, the 2-hydroxyethyl and the 3-hydroxypropyl radicals.

1-4C-Alkylcarbonyl represents a group, which in addition to the carbonyl group contains one of the aforementioned 1-4C-alkyl groups. An example which may be mentioned is the acetyl group.

1-4C-alkoxy-1-4C-alkylcarbonyl represents a group, which in addition to the carbonyl group contains one of the aforementioned 1-4C-alkoxy-1-4C-alkyl groups. Examples which may be mentioned are the methoxymethylcarbonyl (CH3-O—CH2-C(O)—), the ethoxymethylcarbonyl (CH3CH2-O—CH2-C(O)—) and the isobutoxymethylcarbonyl ((CH3)2CH—CH2-O—CH2-C(O)—) group.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

Mono- or di-1-4C-alkylamino-1-4C-alkylcarbonyl represents a 1-4C-alkylcarbonyl group, which is substituted by a mono- or di-1-4C-alkylamino group. Examples, which may be mentioned, are the dimethylamino-methyl-carbonyl ((CH₃)₂N—CH₂—C(O)—) and the diethylamino-methylcarbonyl ((CH₃CH₂)₂N—CH₂—C(O)—) group or the methylamino-methyl-carbonyl (CH₃N(H)—CH₂—C(O)—) group.

For the purpose of the invention, halogen is bromine, chlorine and fluorine.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

The compounds of the formula 1 have at least three centers of chirality in the skeleton. The invention thus provides all feasible enantiomers in any mixing ratio, including the pure enantiomers, which are a preferred subject matter of the invention. One embodiment of the invention (embodiment a) relates to compounds of the formula 1, in which
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R1, R2, R3, R5 and R6 have the meanings as indicated in the outset,
   and the salts of these compounds.

Another embodiment of the invention (embodiment b) relates to compounds of the formula 1, in which
R4 is 1-4C-alkoxy-1-4C-alkylcarbonyl, mono- or di-1-4C-alkylamino-1-4C-alkylcarbonyl or 1-4C-alkylcarbonyl
R1, R2, R3, R5 and R6 have the meanings as indicated in the outset,
   and the salts of these compounds.

A special embodiment of the invention (embodiment c) relates to compounds of the formula 1, in which

R5 is hydrogen

R6 is hydrogen,

R1, R2, R3 and R4 have the meanings as indicated in the outset,

and the salts of these compounds.

The invention also relates to compounds of the formula 1,

in which

R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, halogen, 1-4C-alkyl or fluoro-1-4C-alkyl
R6 is hydrogen, halogen, 1-4C-alkyl or fluoro-1-4C-alkyl
   and the salts of these compounds.

Compounds which are to be mentioned are those of the formula 1 where
R1 is hydrogen, 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, fluoro, 1-4C-alkyl or fluoro-1-4C-alkyl
R6 is hydrogen, fluoro, 1-4C-alkyl or fluoro-1-4C-alkyl
   and the salts of these compounds.

Compounds which are also to be mentioned are those of the formula 1 where
R1 is hydrogen, 1-4C-alkyl or 3-7C-cycloalkyl,
R2 is hydrogen or 1-4C-alkyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl or hydroxy-1-4C-alkyl
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, fluoro, 1-4C-alkyl or fluoro-1-4C-alkyl
R6 is hydrogen, fluoro, 1-4C-alkyl or fluoro-1-4C-alkyl
   and the salts of these compounds.

Compounds which are to be emphasized are those of the formula 1 where
R1 is hydrogen, 1-4C-alkyl or 3-7C-cycloalkyl,
R2 is hydrogen, 1-4C-alkyl, 3-7C-cycloalkyl or 2-4C-alkenyl
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form an ethylen (—CH₂—CH₂—) radical
R5 is hydrogen and
R6 is hydrogen
   and the salts of these compounds.

Compounds which are also to be emphasized are those of the formula 1 where
R1 is hydrogen, 1-4C-alkyl or 3-7C-cycloalkyl,
R2 is hydrogen or 1-4C-alkyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl or hydroxy-1-4C-alkyl
or where R3 and R4 together form an ethylen (—CH₂—CH₂—) radical
R5 is hydrogen and
R6 is hydrogen
   and the salts of these compounds.

Compounds to be particularly emphasized are those of the formula 1, where
R1 is 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 1-4C-alkoxy-1-4C-alkyl or fluoro-1-4C-alkyl,
R4 is hydrogen, 1-4C-alkoxy-1-4C-alkylcarbonyl or mono- or di-1-4C-alkylamino-1-4C-alkylcarbonyl,
or where R3 and R4 together form an ethylen (—CH₂—CH₂—) radical,
R5 is hydrogen,
R6 is hydrogen
   and the salts of these compounds.

Compounds also to be particularly emphasized are those of the formula 1 where
R1 is 1-4C-alkyl,
R2 is 1-4C-alkyl,
R3 is hydrogen, 1-4C-alkyl, 1-4C-alkoxy-1-4C-alkyl or fluoro-1-4C-alkyl,
R4 is hydrogen,
or where R3 and R4 together form an ethylen (—CH₂—CH₂—) radical
R5 is hydrogen,
R6 is hydrogen
   and the salts of these compounds.

Among the compounds of the formula 1 according to the invention, emphasis is given to the optically pure compounds of the formula 1-a 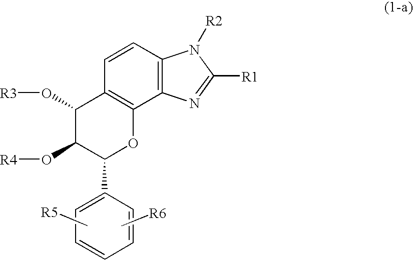
in which R1, R2, R3, R4, R5 and R6 have the meanings as indicated in the outset, and the salts of these compounds.

The invention also relates to compounds of the formula 1-a where
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, halogen, 1-4C-alkyl or fluoro-1-4C-alkyl
R6 is hydrogen, halogen, 1-4C-alkyl or fluoro-1-4C-alkyl
   and the salts of these compounds.

Compounds which are to be mentioned are those of the formula 1-a where
R1 is hydrogen, 1-4C-alkyl or 3-7C-cycloalkyl,
R2 is hydrogen, 1-4C-alkyl, 3-7C-cycloalkyl or 2-4C-alkenyl
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, fluoro, 1-4C-alkyl or fluoro-1-4C-alkyl
R6 is hydrogen, fluoro, 1-4C-alkyl or fluoro-1-4C-alkyl
   and the salts of these compounds.

Compounds which are also to be mentioned are those of the formula 1-a where
R1 is hydrogen, 1-4C-alkyl or 3-7C-cycloalkyl,
R2 is hydrogen or 1-4C-alkyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl or hydroxy-1-4C-alkyl
or where R3 and R4 together form a methylen (—CH₂—), an ethylen (—CH₂—CH₂—), a propylen (—CH₂—CH₂—CH₂—) or a isopropylidene (—C(CH₃)₂—) radical
R5 is hydrogen, fluoro, 1-4C-alkyl or fluoro-1-4C-alkyl
R6 is hydrogen, fluoro, 1-4C-alkyl or fluoro-1-4C-alkyl
   and the salts of these compounds.

Compounds which are to be emphasized are those of the formula 1-a where
R1 is hydrogen, 1-4C-alkyl or 3-7C-cycloalkyl,
R2 is hydrogen, 1-4C-alkyl, 3-7C-cycloalkyl or 2-4C-alkenyl
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form an ethylen (—CH₂—CH₂—) radical
R5 is hydrogen and
R6 is hydrogen
   and the salts of these compounds.

Compounds which are also to be emphasized are those of the formula 1-a where
R1 is hydrogen, 1-4C-alkyl or 3-7C-cycloalkyl,
R2 is hydrogen or 1-4C-alkyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl or hydroxy-1-4C-alkyl
or where R3 and R4 together form an ethylen (—CH₂—CH₂—) radical
R5 is hydrogen and
R6 is hydrogen
   and the salts of these compounds.

Compounds to be particularly emphasized are those of the formula 1-a, where
R1 is 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 1-4C-alkoxy-1-4C-alkyl or fluoro-1-4C-alkyl,
R4 is hydrogen, 1-4C-alkoxy-1-4C-alkylcarbonyl or mono- or di-1-4C-alkylamino-1-4C-alkylcarbonyl,
or where R3 and R4 together form an ethylen (—CH₂—CH₂—) radical,
R5 is hydrogen,
R6 is hydrogen
   and the salts of these compounds.

Compounds also to be particularly emphasized are those of the formula 1-a where
R1 is 1-4C-alkyl,
R2 is 1-4C-alkyl,
R3 is hydrogen, 1-4C-alkyl, 1-4C-alkoxy-1-4C-alkyl or fluoro-1-4C-alkyl,
R4 is hydrogen,
or where R3 and R4 together form an ethylen (—CH₂—CH₂—) radical
R5 is hydrogen,
R6 is hydrogen
   and the salts of these compounds.

Particularly preferred are the compounds given as final products of formula 1 in the examples, and the salts of these compounds.

The compounds according to the invention can be synthesized from corresponding starting compounds, for example according to the reaction schemes given below. The synthesis is carried out in a manner known to the expert, for example as described in more detail in the following examples.

The compounds of formula 1 can be prepared for example by following one of the general reaction sequences designated as route 1 and route 2 as shown in scheme 1.

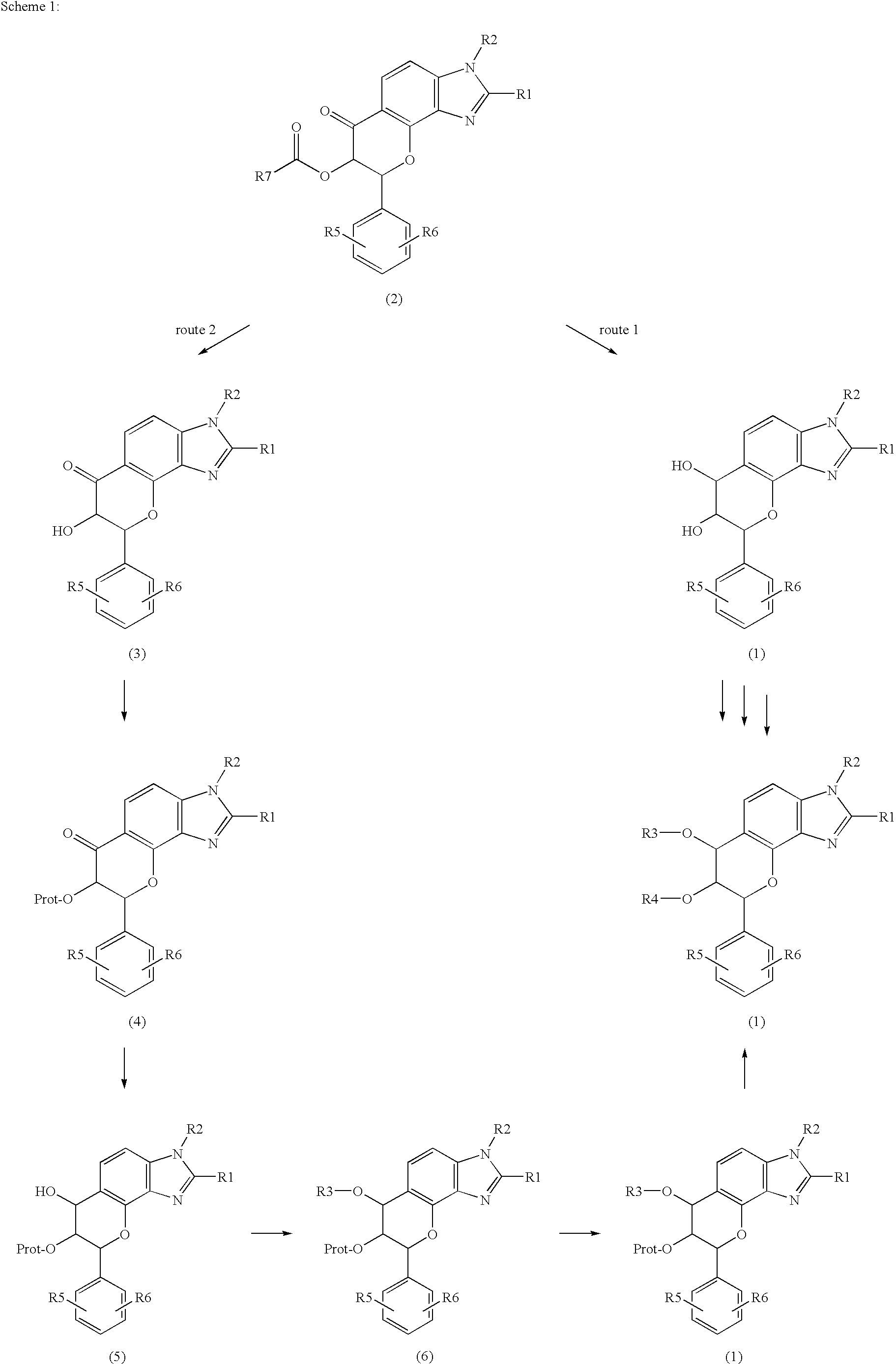

In the reaction sequence designated as route 1, the starting compounds of the formula 2, wherein R7 is for example hydrogen or a 1-4C-alkyl radical, are reduced under conditions which are familiar to the person skilled in the art (e.g. using sodium borohydride derivatives) to yield diols of the general formula 1, wherein R3 and R4 is hydrogen. These compounds can be converted by derivatization reactions which are familiar to the experts (e.g. by alkylation and/or acylation) to give compounds of formula 1 wherein R3 and/or R4 is unequal hydrogen.

In an alternative reaction sequence designated as route 2, the starting compounds of the formula 2 are transformed to compounds of the general formula 3 by methods known to the expert, for example under acidic conditions. The hydroxy functionality in compounds of the formula 3 can be protected by using conditions which are familiar to the person skilled in the art with a protecting group Prot, which preferably does not have an acidic proton, like for example a pivaloyl group, to yield compounds of formula 4. These compounds are selectively reduced under standard conditions, for example using sodium borohydride, to give compounds of formula 5 which are transformed by reaction with suitable derivatization reagents to compounds of the formula 6. After deprotection of the reaction products by methods known to the person skilled in the art, compounds of the formula 1 wherein R4 is hydrogen are obtained. The final compounds of formula 1 with R3 and/or R4 unequal hydrogen are provided by further derivatization reactions which are known to the expert.

Route 2 is the preferred reaction sequence, especially if particular stereoisomers of the final compounds of the formula 1 are desired. The reaction step from compounds of the formula 5 to compounds of the formula 6 can be conducted in a stereochemically selective way by choice of a suitable protection group Prot, like for example a pivaloyl group (see also Scheme 4).

The compounds of formula 1, where R3 and R4 together form a methylen (—CH₂—), an ethylen (—CH₂—CH₂—) or a propylen (—CH₂—CH₂—CH₂—) radical, are designated as compounds of the formula 1* and can be prepared for example by following the reaction sequence shown in scheme 2 (for n 0, 1, 2).

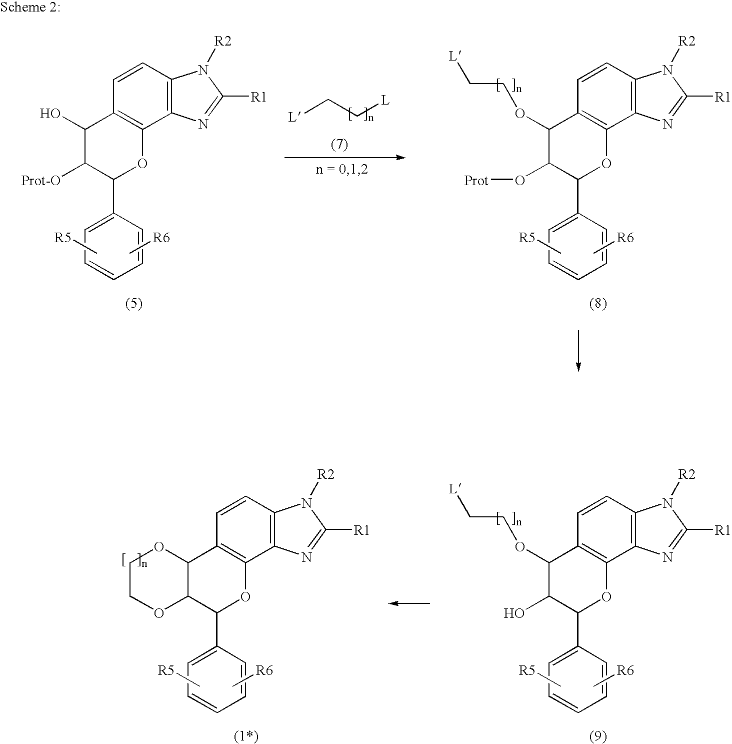

Compounds of the formula 5 are reacted with a compound of the formula 7 to which two leaving groups L and L′ are attached, like for example L=triflate radical and L′=halogen atom, like for example a fluorine atom. The resulting compounds of the formula 8 can be converted to compounds of the formula 9 by methods known to the expert, and the final cyclization reaction to compounds of the formula 1* is likewise carried out in a known manner known per se, for example after reaction with a base, like for example sodium hydride.

Compounds of the formula 2 can be prepared for example as outlined in the following Scheme 3.

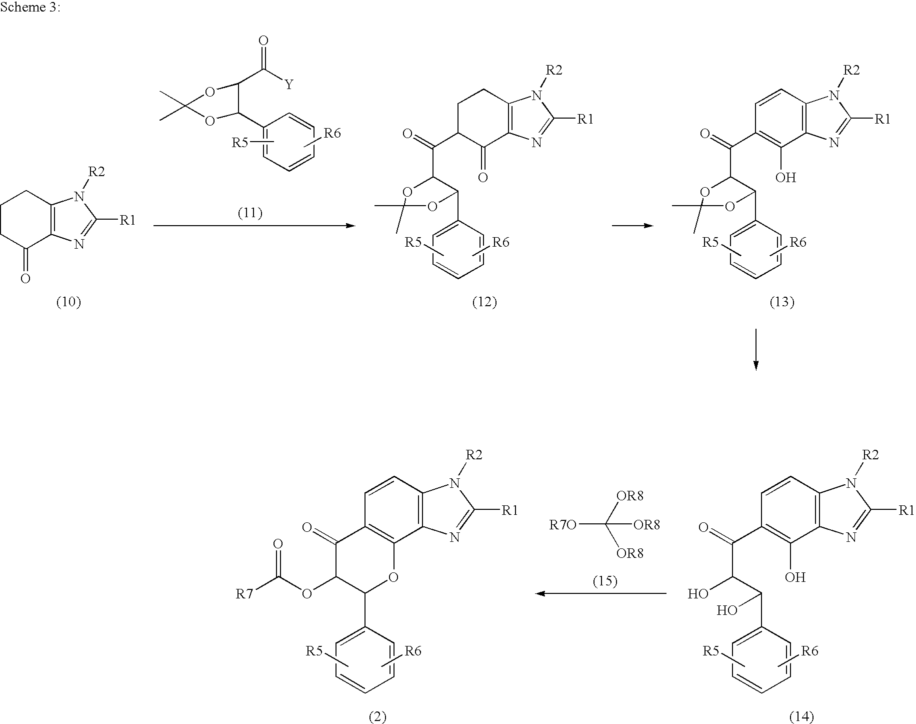

Ketons of the formula 10 can be transformed under basic conditions to compounds of formula 12 by using activated acids of the formula 11, wherein Y is for example a imidazole, chloride, bromide or a 1-4C-alkoxy radical. The compounds of the formula 12 are oxidized under standard conditions. Deprotection, for example under acidic conditions, of the resulting compounds of the formula 13 leads to triols of the general formula 14. These triols are cyclized under acidic conditions, for example using ortho-esters of the formula 15, wherein R7 is for example hydrogen or 1-4C-alkyl and R8 is for example 1-4C-alkyl, to give the desired compounds of the formula 2.

The syntheses as shown in Schemes 1, 2 or 3 can provide particular stereoisomers or mixtures of selected, particular stereoisomers of compounds of the formula 1, for example by starting from one stereoisomer of the compounds of the formula 11. By appropriate selection of the stereochemistry of the compounds of the formula 11, each stereoisomer of the final compounds of the formula 1 can be synthesized. As an example, the emphasized optically pure compounds of the formula 1-a can be obtained by following the reaction sequences described above in Scheme 3 and in Scheme 1 (preferably route 1) or Scheme 2. by starting from compounds of the formula 11-a as shown in abbreviated form in Scheme 4.

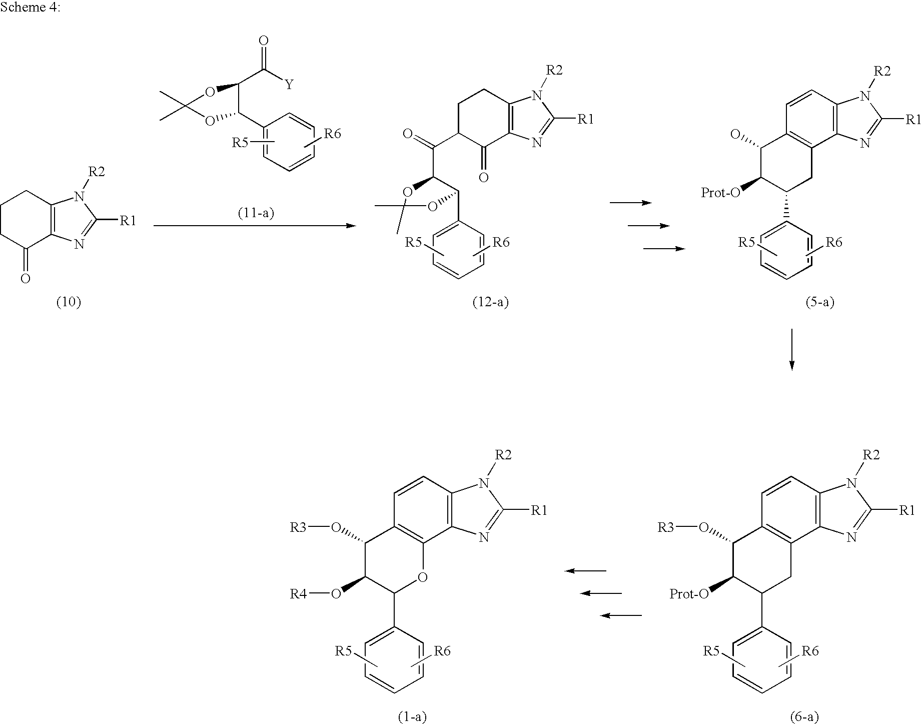

Ketones of the formula 10 are known, for example from Helvetica Chimica Acta (1979), 62, 507, or can be prepared in a manner as shown for example in scheme 5 (route A). 3-Nitro-2-aminophenol can be reacted in a first step with a suitable benzyl derivative, for example benzylchloride, and the amino group of the reaction product of the formula 17 (known from J. Heterocyclic Chem. (1983), 20, 1525) is converted to the di-amide of the formula 18. Subsequent reduction under standard conditions, for example using hydrazine N₂H₄ in the presence of FeCl₃, leads to the formation of the primary amide of the formula 19, whose amine functionality can be alkylated in a next step, for example under reductive alkylation conditions, to compounds of the formula 20. The following cyclization step is performed under standard conditions, for example under acidic conditions using POCl₃, to give compounds of the formula 21 whose hydrogenation to the desired compounds of the formula 10 is performed in manner known to the expert, for example as described by H. Oelsohlaeger and H. Giebenhain in Archiv der Pharmazie, 1973, 306, 485-489.

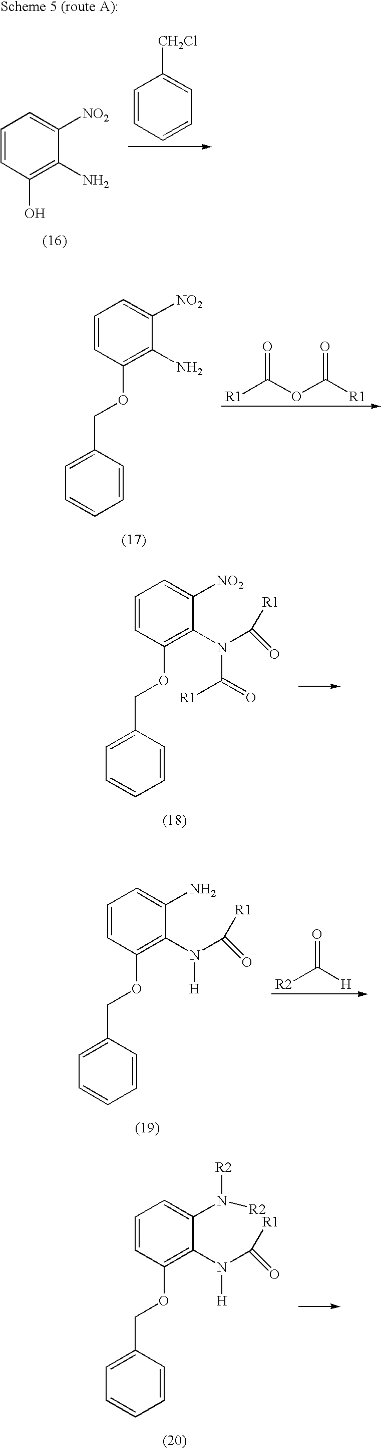

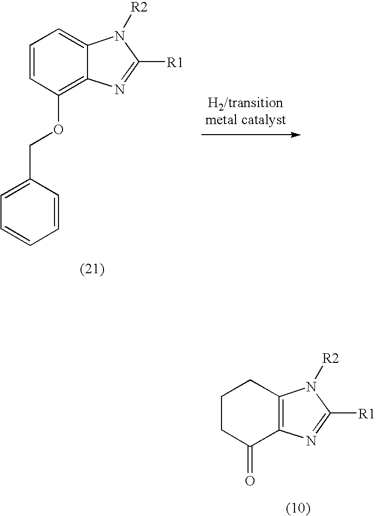

Alternatively, the ketones of the formula 10 can be prepared from compounds of the formula 25 by a cydization reaction in the presence of a primary amine as shown in scheme 6 (route B). Compounds of the formula 25 are known, for example from H. Stetter and K. Hoehne, Chem. Ber., 1958, 91, 1123-1128, or can be prepared in an analogous manner starting from 2-nitroresorcin as shown in scheme 6.

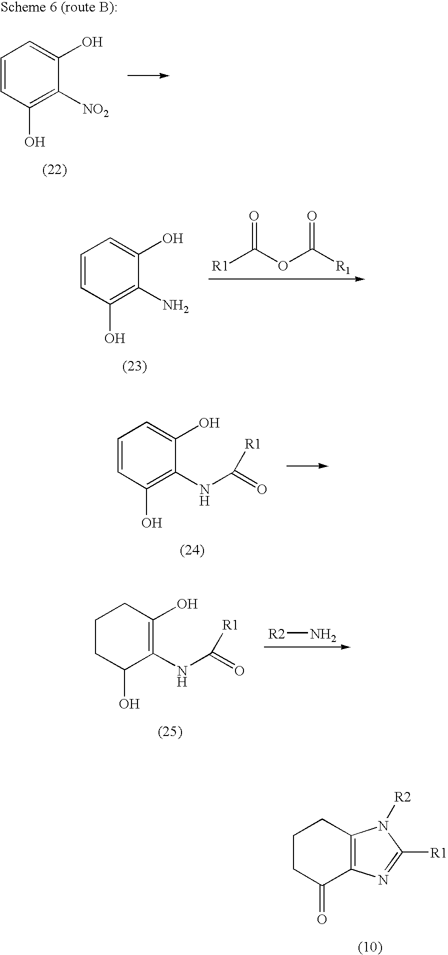

### ADVANTAGEOUS EFFECTS

The excellent gastric protective action and the gastric acid secretion-inhibiting action of the compounds according to the invention can be demonstrated in investigations on animal experimental models. The compounds of the formula 1 according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 1 | 2 | >50 |
| 2 | 2 | <50 |
| 3 | 2 | <50 |
| 4 | 2 | >50 |
| 5 | 2 | >50 |
| 6 | 2 | >50 |
| 7 | 2 | >50 |
| 8 | 2 | >50 |
| 9 | 2 | >50 |
| 11 | 2 | <50 |
| 15 | 2 | >50 |
| 16 | 2 | >50 |
| 17 | 2 | <50 |
| 18 | 2 | <50 |
| 19 | 2 | >50 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

### MODE(S) FOR CARRYING OUT THE INVENTION

The examples below serve to illustrate the invention in more detail without limiting it. Further compounds of the formula 1 whose preparation is not described explicitly can likewise be prepared in an analogous manner or in a manner known per se to the person skilled in the art, using customary process techniques. The compounds named expressly as examples, and the salts of these compounds, are preferred subject matter of the invention. The abbreviation min stands for minute(s), h stands for hour(s), m.p. stands for melting point and ee for enantiomeric excess.

I. Final Compounds of the Formula 1

### 1. 1.(6R,7S,8R)-7-Hydroxy-6-(2-methoxy-ethoxy)-2,3-dimethyl-8-phenyl-3,6,7,8-tetrahydro-chromeno[7,8-d]imidazole

To a suspension of 0.82 g (2.64 mmol) (6R,7S,8R)-6,7-dihydroxy-2,3-dimethyl-8-phenyl-3,6,7,8-tetrahydro-chromeno[7,8-d]imidazole in 2-methoxyethanol were added 0.32 ml (6.0 mmol) conc. sulphuric acid. The mixture was stirred for 3 h at 120° C. The reaction was quenched by pouring out into a saturated sodium hydrogen carbonate solution and extracted with ethyl acetate three times. The combined organic layers were concentrated in vacuo and purified by column chromatography (dichloromethane/methanol:100/3). The product was crystallized from ethyl acetate to give 0.25 g (0.68 mmol/26%) of the title product as a colourless solid with a melting point of 206° C. (ethyl acetate).

### 2. (6S,7S,8R)-7-Hydroxy-6-(2-methoxy-ethoxy)-2,3-dimethyl-8-phenyl-3,6,7,8-tetrahydro-chromeno[7,8-d]imidazole

To a suspension of 0.82 g (2.64 mmol) (6R,7S,8R)-6,7-dihydroxy-2,3-dimethyl-8-phenyl-3,6,7,8-tetrahydro-chromeno[7,8-d]imidazole in 2-methoxyethanol were added 0.32 ml (6.0 mmol) conc. sulphuric acid. The mixture was stirred for 3 h at 120° C. The reaction was quenched by pouring out into a saturated sodium hydrogen carbonate solution and extracted with ethyl acetate three times. The combined organic layers were concentrated in vacuo and purified by column chromatography (dichloromethane/methanol:100/3). The product was crystallized from acetone to give 0.11 g (0.29 mmol/11%) of the title product as a colourless solid with a melting point of 163° C. (ethyl acetate).

### 3. (6R,7S,8R)-6,7-Dihydroxy-2,3-dimethyl-8-phenyl-3,6,7,8-tetrahydro-chromeno[7,8-d]imidazole

To a stirred suspension of 1.30 g (3.70 mmol) (7R,8R)-7-acetoxy-2,3-dimethyl-8-phenyl-7,8-dihydro-3H-chromeno[7,8-d]imidazol-6-one in methanol (25 ml) were added 0.45 g (11.4 mmol) sodium boron hydride and it was stirred for further 1 h. Subsequently the reaction was quenched by pouring out into a saturated ammonium chloride solution. The mixture was extracted with dichloromethane three times. The combined organic layers were concentrated in vacuo and purified by column chromatography (dichloromethane/methanol:13/1) to give 1.10 g (3.54 mmol/95%) of the title product as a colourless solid with a melting point of 262.5° C. (dichloromethane/methanol).

### 4. (6R,7S,8R)-6-Ethoxy-7-hydroxy-2,3-dimethyl-8-phenyl-3,6,7,8-tetrahydro-chromeno[7,8-d]imidazole

To a solution of 1.80 g (4.26 mmol) (6R,7S,8R)-6-ethoxy-2,3-dimethyl-8-phenyl-7-pivaloxy-7,8-dihydro-3H-chromeno[7,8-d]imidazol-6-one in methanol (15 ml) were added 1.20 g (8.52 mmol) potassium carbonate and the mixture was stirred for 20 h. Afterwards the reaction was quenched by pouring out into a saturated ammonium chloride solution. This mixture was extracted with dichloromethane three times and the combined organic layers were concentrated in vacuo. The crude product was purified by column chromatography (dichloromethane/methanol: 100/3) to give 1.17 g (3.45 mmol/81%) of the title product as a colourless solid with a melting point of 222° C. (dichloromethane/methanol).

### 5. (6R,7S,8R)-6-(2-Fluoro-ethoxy)-7-hydroxy-2,3-dimethyl-8-phenyl-3,6,7,8-tetrahydro-chromeno[7,8-d]imidazole

To a solution of 1.20 g (2.72 mmol) (6R,7S,8R)-6-(2-fluoro-ethoxy)-2,3-dimethyl-8-phenyl-7-pivaloxy-7,8-dihydro-3H-chromeno[7,8-d]imidazol-6-one in methanol (12 ml) were added 0.40 g (2.90 mmol) potassium carbonate and the mixture was stirred for 20 h. Afterwards the reaction was quenched by pouring out into a saturated ammonium chloride solution. This mixture was extracted with dichloromethane three times and the combined organic layers were concentrated in vacuo. The crude product was purified by column chromatography (dichloromethane/methanol:100/3) to give 0.95 g (2.67 mmol/98%) of the title product as a colourless solid with a melting point of 226° C. (dichloromethane/methanol).

### 6. (5S,6R,10R)-16,17-Dimethyl-6-phenyl-2,3,5,6,10,17-hexahydro-1,4,7-trioxa-15,17-diaza-cyclopenta[a]phenanthrene

A suspension of 0.60 g (1.68 mmol) (6R,7S,8R)-6-(2-fluoro-ethoxy)-7-hydroxy-2,3-dimethyl-8-phenyl-3,6,7,8-tetrahydro-chromeno[7,8-d]imidazole and 0.60 g (15.0 mmol) sodium hydride (60% dispersion in mineral oil) in dimethyl formamide (12 ml) was stirred for 1.5 h at 50° C. The reaction was quenched by pouring out into a saturated sodium hydrogen carbonate solution and extracted with ethyl acetate three times. The combined organic layers were concentrated in vacuo and purified by column chromatography (dichloromethane/methanol:100/3). The product was crystallized from ethyl acetate to give 0.43 g (1.28 mmol/76%) of the title product as a colourless solid with a melting point of 307° C. (ethyl acetate).

### 7. (6R,7S,8R)-7-(2-Methoxy-acetoxy)-6-(2-methoxy-ethoxy)-2,3-dimethyl-8-phenyl-3,6,7,8-tetrahydro-chromeno[7,8-d]imidazole

To a suspension of 0.60 g (1.63 mmol) (6R,7S,8R)-7-hydroxy-6-(2-methoxy-ethoxy)-2,3-dimethyl-8-phenyl-3,6,7,8-tetrahydro-chromeno[7,8-d]imidazole in dichloro-methane (12 ml) is added 1.25 ml (5.00 mmol) triethylamine, 20.0 mg (0.16 mmol) 4-dimethylaminopyridine and 0.56 g (5.00 mmol) methoxyacetyl chloride diluted in dichloromethane (2 ml). The reaction is stirred for further 4 h at 25° C. Subsequently the mixture is poured out into water and extracted with dichloromethane three times. The combined organic layers are concentrated in vacuo and purified by column chromatography (dichloromethane/methanol: 100/3). The product is crystallized from ethyl acetate to give 0.09 g (0.20 mmol/13%) of the title product as a colourless solid with a melting point of 200° C. (ethyl acetate).

### 8. (6R,7S,8R)-7-(2-Dimethylamino-acetoxy)-6-(2-methoxy-ethoxy)-2,3-dimethyl-8-phenyl-3,6,7,8-tetrahydro-chromeno[7,8-d]imidazole

To a suspension of 0.60 g (1.63 mmol) (6R,7S,8R)-7-hydroxy-6-(2-methoxy-ethoxy)-2,3-dimethyl-8-phenyl-3,6,7,8-tetrahydro-chromeno[7,8-d]imidazole in dichloro-methane (12 ml) is added 1.25 ml (5.00 mmol) triethylamine, 20.0 mg (0.16 mmol) 4-dimethylaminopyridine and 0.40 g (2.23 mmol) dimethylaminoacetic acid chloride hydrochloride. The reaction is stirred for further 6 d at 25° C. Subsequently the mixture is poured out into water and extracted with dichloromethane three times. The combined organic layers are concentrated in vacuo and purified by column chromatography (dichloromethane/methanol: 100/3). The product is reslurried from diethyl ether to give 0.52 g (1.15 mmol/70%) of the title product as a colourless solid with a melting point of 175° C. (diethyl ether).

### 9. (6R,7S,8R)-7-Hydroxy-6-(2,2-difluoro-ethoxy)-2,3-dimethyl-8-phenyl-3,6,7,8-tetrahydro-chromeno[7,8-d]imidazole

To a at 0° C. cooled solution of 0.90 g (1.96 mmol) (6R,7S,8R)-6-(2,2-Difluoro-ethoxy)-2,3-dimethyl-8-phenyl-7-pivaloxy-3,6,7,8-tetrahydro-chromeno[7,8-d]imidazole in methanol (10 ml) is added 0.54 g (3.91 mmol) potassium carbonate and the reaction is stirred for further 2 d at 25° C. The reaction is quenched by pouring out into a saturated ammonium chloride solution and extracted with dichloromethane three times. The combined organic layers are concentrated in vacuo and purified by column chromatography (dichloromethane/methanol:100/1). to give 0.57 g (1.52 mmol/78%) of the title product as a colourless solid with a melting point of 249° C. (dichloromethane/methanol).

### 10. (6R,7S,8R)-6,7-Dihydroxy-2-methyl-8-phenyl-3,6,7,8-tetrahydro-chromeno[7,8-d]imidazole

To a stirred suspension of 0.50 g (1.33 mmol) (7R,8R)-7-acetoxy-2,-methyl-8-phenyl-7,8-dihydro-3H-chromeno[7,8-d]imidazol-6-one in methanol (10 ml) is added 0.15 g (3.98 mmol) sodium borohydride and it is stirred for further 1 h. Subsequently the reaction is quenched by pouring out into a saturated ammonium chloride solution. The mixture is extracted with dichloromethane/methanol (13/1) ten times. The combined organic layers are concentrated in vacuo to give 0.35 g (1.18 mmol/89%) of the title product as a colourless solid with a melting point of 163° C. (dichloromethane/methanol).

### 11. (6R,7S,8R)-3Allyl-7-hydroxy-6-(2-methoxy-ethoxy)-2-methyl-8-phenyl-3,6,7,8-tetrahydro-chromeno[7,8-d]imidazole

To a at 0° C. cooled solution of 0.04 g (0.08 mmol) (6R,7S,8R)-3-allyl-6-(2-methoxy-ethoxy)-2-methyl-8-phenyl-7-pivaloxy-3,6,7,8-tetrahydro-chromeno[7,8-d]imidazole in methanol (1 ml) is added 0.01 g (0.08 mmol) potassium carbonate and the reaction is stirred for further 6 d at 25° C. The reaction is quenched by pouring out into a saturated ammonium chloride solution and extracted with dichloromethane three times. The combined organic layers are concentrated in vacuo and purified by column chromatography (dichloromethane/methanol:100/1). to give 0.03 g (0.08 mmol/98%) of the title product as a colourless solid with a melting point of 79° C. (dichloromethane/methanol).

### 12. (6R,7S,8R)-7-Hydroxy-6-(2-methoxy-ethoxy)-2-methyl-8-phenyl-3-propenyl-3,6,7,8-tetrahydro-chromeno[7,8-d]imidazole*1

To a at −40° C. cooled suspension of 4.30 g (10.2 mmol) (6R,7S,8R)-6-hydroxy-2-methyl-8-phenyl-7-pivaloxy-3-propenyl-3,6,7,8-tetrahydro-3H-chromeno[7,8-d]imidazole in THF (86 ml) is added 2.20 g (10.6 mmol) 2-methoxyethyl triflate and 23.8 ml (23.8 mmol) bis-(trimethylsilyl)-sodium amide (1 M in THF). This mixture is stirred for 1 h at −40° C. Subsequently the reaction is quenched by pouring out into saturated ammonium chloride solution and is extracted with dichloromethane three times. The combined organic layers are concentrated in vacuo and purified by column chromatography (dichloromethane/methanol:100/3) to give 0.80 g (2.01 mmol/20%) of the title product as a colourless solid with a melting point of 61° C.*1 (dichloromethane/methanol).

*1=The product is a mixture of E/Z-isomeres (2/1) in 3-propenyl.

### 13. (6R,7S,8R)-7-Hydroxy-6-(2-methoxy-ethoxy)-2-methyl-8-phenyl-3,6,7,8-tetrahydro-chromeno[7,8-d]imidazole

To a at 0° C. cooled solution of 0.40 g (0.92 mmol) (6R,7S,8R)-6-(2-methoxy-ethoxy)-2-methyl-8-phenyl-7-pivaloxy-3,6,7,8-tetrahydro-chromeno[7,8-d]imidazole in methanol (9 ml) is added 0.12 g (0.92 mmol) potassium carbonate and the reaction is stirred for further 2.5 d at 25° C. The reaction is quenched by pouring out into a saturated ammonium chloride solution and extracted with dichloromethane four times. The combined organic layers are concentrated in vacuo and purified by column chromatography (dichloromethane/methanol:100/3). to give 0.20 g (0.56 mmol/62%) of the title product as a colourless solid with a melting point of 117° C. (dichloromethane/methanol).

### 14. (6R,7S,8R)-3-Allyl-7-hydroxy-6-(2-fluoro-ethoxy)-2-methyl-8-phenyl-3,6,7,8-tetrahydro-chromeno[7,8-d]imidazole

To a at 0° C. cooled solution of 6.70 g (14.4 mmol) (6R,7S,8R)-6-(2-fluoro-ethoxy)-2-methyl-8-phenyl-7-pivaloxy-3,6,7,8-tetrahydro-chromeno[7,8-d]imidazole in methanol (30 ml) is added 2.00 g (14.5 mmol) potassium carbonate and the reaction is stirred for further 3 d at 25° C. The reaction is quenched by pouring out into a saturated ammonium chloride solution and extracted with dichloromethane three times. The combined organic layers are concentrated in vacuo and purified by column chromatography (dichloromethane/methanol:100/1). to give 4.74 g (12.4 mmol/86%) of the title product as a colourless solid with a melting point of 117° C. (dichloromethane/methanol).

### 15. (5S,6R,10R)-16-Methyl-6-phenyl-17-propenyl-2,3,5,6,10,17-hexahydro-1,4,7-trioxa-15,17-diaza-cyclopenta[a]phenanthrene*1

To a solution of 3.20 g (8.37 mmol) (6R,7S,8R)-3-allyl-7-hydroxy-6-(2-fluoro-ethoxy)-2-methyl-8-phenyl-3,6,7,8-tetrahydro-chromeno[7,8-d]imidazole in dimethyl form-amide (70 ml) is added 800 mg (20.0 mmol) sodium hydride (60% dispersion in mineral oil) and the reaction mixture is stirred for 1.5 h at 50° C. The reaction is quenched by pouring out into a saturated ammonium chloride solution and the precipitated crude product is filtered off, dried and purified by column chromatography (dichloromethane/methanol: 100/1). The product is crystallized from diethyl ether to give 1.13 g (3.12 mmol/38%) of the title product as a colourless solid with a melting point of 213° C.*1 (diethyl ether).

1H-NMR (200 MHz, CDCl3): δ=1.55 (dd, 3H), 2.48 (s, 3H), 3.81-4.00 (m, 5H), 4.85 (d, 1H), 5.20 (d, 1H), 6.00 (q, 1H), 6.56 (dd, 1H), 6.82 (d, 1H), 7.28 (d, 1H), 7.38-7.45 (m, 3H), 7.53-7.58 (m, 2H).

*1=The product is a mixture of E/Z-isomeres (95/5) in 3-propenyl

### 16. (5S,6R,10R)-16-Methyl-6-phenyl-2,3,5,6,10,17-hexahydro-1,4,7-trioxa-15,17-diaza-cyclopenta[a]phenanthrene

To a stirred solution of 1.25 g (3.45 mmol) of (5S,6R,10R)-16-methyl-6-phenyl-17-propenyl-2,3,5,6,10,17-hexahydro-1,4,7-trioxa-15,17-diaza-cyclopenta[a]phenanthrene in acetone (22 ml) is added 2.30 g (14.4 mmol) potassium permanganate and it is stirred for further 2 h at 25° C. Afterwards the reaction mixture is filtered off and the filtrate is concentrated in vacuo. The crude product is purified by column chromatography (dichloromethane/methanol:100/3). The product is crystallized from diethyl ether to give 0.27 g (0.84 mmol/24%) of the title product as a colourless solid with a melting point of 260° C. (diethyl ether).

### 17. (6R,7S,8R)-3-Cyclopropyl-6,7-dihydroxy-2-methyl-8-phenyl-3,6,7,8-tetrahydro-chromeno[7,8-d]imidazole

To a stirred suspension of 6.00 g (18.0 mmol) (7R,8R)-3-cyclopropyl-7-hydroxy-2-methyl-8-phenyl-7,8-dihydro-3H-chromeno[7,8-d]imidazol-6-one in methanol (115 ml) is added 0.90 g (24.0 mmol) sodium borohydride and it is stirred for further 4 h. Subsequently the reaction is quenched by pouring out into a saturated ammonium chloride solution. The mixture is extracted with dichloromethane three times. The combined organic layers are concentrated in vacuo and purified by column chromatography (dichloromethane/methanol: 100/1) to give 5.26 g (15.6 mmol/87%) of the title product as a colourless solid.

1H-NMR (200 MHz, CDCl3): δ=0.97-1.05 (m, 2H), 1.16-1.26 (m, 2H), 2.57 (s, 3H), 3.10-3.23 (m, 1H), 3.97 (dd, 1H), 4.93 (d, 2H), 7.13 (d, 1H), 7.26-7.37 (m, 4H), 7.43-7.50 (m, 2H).

### 18. (6R,7S,8R)-3-Cyclopropyl-7-hydroxy-6-(2-methoxy-ethoxy)-2-methyl-8-phenyl-3,6,7,8-tetrahydro-chromeno[7,8-d]imidazole

To a suspension of 4.10 g (12.2 mmol) (6R,7S,8R)-3-cyclopropyl-6,7-dihydroxy-2-methyl-8-phenyl-3,6,7,8-tetrahydro-chromeno[7,8-d]imidazole in 2-methoxyethanol (41 ml) is added 1.64 ml (30.7 mmol) conc. sulphuric acid. The mixture is stirred for 1.5 h at 100° C. The reaction is quenched by pouring out into a saturated sodium hydrogen carbonate solution and extracted with dichloromethane four times. The combined organic layers are concentrated in vacuo and purified by column chromatography (dichloromethane/methanol: 100/1) and triethylamine (100). The product is crystallized from ethyl acetate to give 0.23 g (0.58 mmol/5%) of the title product as a colourless solid with a melting point of 183° C. (ethyl acetate).

### 19. (6S,7S,8R)-3-Cyclopropyl-7-hydroxy-6-(2-methoxy-ethoxy)-2-methyl-8-phenyl-3,6,7,8-tetrahydro-chromeno[7,8-d]imidazole

To a suspension of 4.10 g (12.2 mmol) (6R,7S,8R)-3-cyclopropyl-6,7-dihydroxy-2-methyl-8-phenyl-3,6,7,8-tetrahydro-chromeno[7,8-d]imidazole in 2-methoxyethanol (41 ml) is added 1.64 ml (30.7 mmol) conc. sulphuric acid. The mixture is stirred for 1.5 h at 100° C. The reaction is quenched by pouring out into a saturated sodium hydrogen carbonate solution and extracted with dichloromethane four times. The combined organic layers are concentrated in vacuo and purified by column chromatography (dichloromethane/methanol:100/1) and triethylamine (100). The product is crystallized from ethyl acetate to give 0.62 g (1.57 mmol/13%) of the title product as a colourless solid with a melting point of 218° C. (ethyl acetate).

### 20. (6R,7S,8R)-2-Cyclopropyl-6,7-dihydroxy-3-methyl-phenyl-3,6,7,8-tetrahydro-chromeno[7,8-d]imidazole

To a stirred suspension of 3.00 g (7.97 mmol) (7R,8R)-2-cyclopropyl-7-hydroxy-3-methyl-8-phenyl-7,8-dihydro-3H-chromeno[7,8-d]imidazol-6-one in methanol (60 ml) is added 0.61 g (16.1 mmol) sodium borohydride and it is stirred for further 0.5 h. Subsequently the reaction is quenched by pouring out into a saturated ammonium chloride solution. The mixture is extracted with dichloromethane four times. The combined organic layers are concentrated in vacuo and purified by column chromatography (dichloromethane/methanol:100/1) to give 2.45 g (7.28 mmol/92%) of the title product as a colourless foam.

1H-NMR (200 MHz, CDCl3): δ=0.99-1.29 (m, 4H), 1.86-200 (m, 1H), 3.80 (s, 3H), 3.98 (dd, 1H), 4.96 (t, 2H), 6.94 (d, 1H), 7.19-7.39 (m, 4H), 7.49-7.53 (m, 2H).

### 21. (6R,7S,8R)-2-Cyclopropyl-7-hydroxy-6-(2-methoxy-ethoxy)-3-methyl-8-phenyl-3,6,7,8-tetrahydro-chromeno[7,8-d]imidazole

To a suspension of 2.40 g (7.13 mmol) (6R,7S,8R)-2-cyclopropyl-6,7-dihydroxy-3-methyl-8-phenyl-3,6,7,8-tetrahydro-chromeno[7,8-d]imidazole in 2-methoxyethanol (24 ml) is added 0.90 ml (16.9 mmol) conc. sulphuric acid. The mixture is stirred for 1.5 h at 100° C. The reaction is quenched by pouring out into a saturated sodium hydrogen carbonate solution and extracted with dichloromethane three times. The combined organic layers are concentrated in vacuo and purified by column chromatography (dichloromethane/methanol: 100/3) and triethylamine (100). The product is crystallized from ethyl acetate to give 0.49 g (1.24 mmol/18%) of the title product as a colourless solid with a melting point of 157° C. (ethyl acetate).

### 22. (6S,7S,8R)-2-Cyclopropyl-7-hydroxy-6-(2-methoxy-ethoxy)-3-methyl-8-phenyl-3,6,7,8-tetrahydro-chromeno[7,8-d]imidazole

To a suspension of 2.40 g (7.13 mmol) (6R,7S,8R)-2-cyclopropyl-6,7-dihydroxy-3-methyl-8-phenyl-3,6,7,8-tetrahydro-chromeno[7,8-d]imidazole in 2-methoxyethanol (24 ml) is added 0.90 ml (16.9 mmol) conc. sulphuric acid. The mixture is stirred for 1.5 h at 100° C. The reaction is quenched by pouring out into a saturated sodium hydrogen carbonate solution and extracted with dichloromethane three times. The combined organic layers are concentrated in vacuo and purified by column chromatography (dichloromethane/methanol:100/3) and triethylamine (100). The product is crystallized from ethyl acetate to give 0.98 g (2.48 mmol/35%) of the title product as a colourless solid with a melting point of 133° C. (ethyl acetate).

II. Starting Compounds

### A. 2-Benzyloxy-6-nitroaniline

To a solution of 50.0 g (0.31 mol) of 2-amino-3-nitrophenol in ethanol (400 ml) were added 43.5 ml (0.38 mol) benzyl chloride, 47.8 g (0.35 mol) potassium carbonate and 2.00 g (13.3 mmol) sodium iodide and it was stirred at 80° C. for 3.5 h. Subsequently the mixture was concentrated in vacuo, re-dissolved in dichloromethane, washed with water, dried over sodium sulfate, filtrated over sand and concentrated in vacuo again. The crude product was purified by column chromatography (cyclohexane/ethyl acetate: 8/2) to give 76.0 g (0.31 mol/96%) of the title product.

1H-NMR (200 MHz, CDCl₃): δ=5.11 (s, 2H), 6.57 (t, 1H), 6.95 (d, 1H), 7.35-7.44 (m, 5H), 7.73 (d, 1H).

### B. N-Acetyl-2-benzyloxy-6-nitro-acetanilide

To a stirred reaction mixture of 76.0 g (0.31 mol) 2-benzyloxy-6-nitroaniline in acetic anhydride (469 ml) was added 7.60 ml (0.12 mol) methane sulfonic acid and stirred for 2 h at 120° C. Afterwards the acetic anhydride was removed in vacuo and the residue was poured into ice water. This mixture was neutralised with concentrated ammonia solution and extracted with dichloromethane three times. The combined organic layers were concentrated and dried in vacuo to give 99.9 g (0.30 mol/98%) of the title product with a melting point of 113.8° C. (dichloromethane).

### C. 2-Amino-6-benzyloxy-acetanilide

To a stirred mixture of 99.6 g (0.30 mol) N-acetyl-2-benzyloxy-6-nitro-acetanilide, activated carbon (59.7 g) and 30.0 g (18.5 mmol) iron chloride in methanol (2.60 l) at 70° C. was added dropwise 147 ml (3.03 mol) hydrazine hydrate and stirred for further 5 h. Subsequently the mixture was filtrated over kieselgur and concentrated in vacuo. The crude mixture was suspended in a saturated ammonium chloride solution and extracted with dichloromethane twice. The combined organic layers were concentrated in vacuo and the crude product was reslurried in diethyl ether to give 50.5 g (0.20 mol/65%) of the title product with a melting point of 146.9° C. (diethyl ether).

### D. 4-Benzyloxy-1,2-dimethyl-1H-benzimidazole

To a stirred mixture of 4.00 g (17.0 mmol) 2-amino-6-benzyloxy-acetanilide in dichloromethane (8.0 ml) were added 4.00 ml (4.30 mmol) phosphoryl chloride and the mixture was stirred at 70° C. for 5 h. Subsequently the mixture was poured into ice water, neutralised by adding sodium hydroxide solution (6 N) and extracted with dichloromethane three times. The combined organic layers were concentrated in vacuo and the crude product was purified by column chromatography (diethyl ether/petrol ether 7/3) to give 3.09 g (12.2 mmol/72%) of the title product with a melting point of 130.9° C. (diethyl ether/petrol ether).

### E. 1,2-Dimethyl-1,5,6,7-tetrahydro-benzoimidazol-4-one

Route A: A suspension of 2.00 g (7.93 mmol) 4-benzyloxy-1,2-dimethyl-1H-benzimidazole and 1.70 g palladium on carbon (10%) in methanol (50 ml) was stirred in an autoclave under a hydrogen pressure of 150 bar at 70° C. for 20 h. Afterwards the catalyst was filtered off and the methanol was removed in vacuo. The crude product was purified by column chromatography (dichloromethane/methanol: 100/3 to 13/1) to give 0.14 g (0.85 mmol/11%) of the title product with a melting point of 98.1° C. (dichloromethane/methanol).

Route B: To a stirred mixture of 29.0 g (0.17 mol) 2-acetylamino-3-hydroxy-cyclohex-2-enone in xylene (580 ml) were added 57 ml of acetic acid and dropwise 116 ml (0.23 mol) of methylamine (2 M in THF). The reaction mixture was heated to 155° C. for 5 h, cooled down to 25° C. and stirred for further 20 h. Afterwards the mixture was concentrated in vacuo and the crude product was purified by column chromatography (ethyl acetate/methanol:8/2) to yield 21.4 g (0.13 mol/77%) of the title product with a melting point of 98.1° C. (ethyl acetate/methanol).

### F. 5-[1-((4R,5S)-2,2-Dimethyl-5-phenyl-[1,3]dioxolan-4-yl)methanoyl]-1,2-dimethyl-1,5,6,7-tetrahydro-benzoimidazol-4-one

To 19.2 g (0.48 mol) sodium hydride (60% dispersion in mineral oil) was added pentane (200 ml) and this suspension was stirred for 5 min. Afterwards the supernatant pentane was decanted of. This procedure was carried out two times to activate the sodium hydride. To this activated sodium hydride was added THF (1.20 l) and spatula wise 60.0 g (0.37 mol) of freshly oven dried 1,2-dimethyl-1,5,6,7-tetrahydro-benzoimidazol-4-one. This suspension was stirred for 30 min at 25° C. and at 60° C. until the evolution of hydrogen gas stops. At the same time (2R,3S)-2,3-O,O-isopropylidene-3-phenyl-propionic acid was activated by the following procedure:

To a stirred solution of 96.0 g (0.43 mol) (2R,3S)-2,3-O,O-isopropylidene-3-phenyl-propionic acid in THF (250 ml) were added portion wise 73.8 g (0.43 mol) N,N′-carbonyldiimidazole. This solution was stirred for 2 h at 25° C.

Afterwards this solution was added dropwise to the at 60° C. stirred reaction suspension and the mixture was stirred for further 1 h. Subsequently the reaction was quenched by pouring out into a saturated ammonium chloride solution. The mixture is extracted with ethyl acetate three times. The combined organic layers were concentrated in vacuo and purified by column chromatography (dichloromethane/methanol: 100/3) and reslurried in diethyl ether to give 67.3 g (0.18 mol/50%) of the title product as a colourless solid with a melting point of 144° C. (diethyl ether).

### G. 4-Hydroxy-5-[1-((4R,5S)-2,2-dimethyl-5-phenyl-[1,3]dioxolan-4-yl)-methanoyl]-1,2-dimethyl-1H-benzoimidazole

65.0 g (0.18 mol) 5-[1-((4R,5S)-2,2-dimethyl-5-phenyl-[1,3]dioxolan-4-yl)methanoyl]-1,2-dimethyl-1,5,6,7-tetrahydro-benzoimidazol-4-one and 155 g (1.79 mol) manganese(IV)oxide in dioxane (1.30 l) were stirred for 18 h at 25° C. Afterwards the reaction mixture was transferred into a Soxhlet and extracted for 5 h at 160° C. The dioxane solution was concentrated in vacuo and the crude product was reslurried from diethyl ether to give 41.6 g (0.12 mol/64%) of the title product as a colourless solid with a melting point of 233° C. (diethyl ether).

### H. (2R,3S)-2,3-Dihydroxy-1-(4-hydroxy-1,2-dimethyl-1H-benzoimidazol-5-yl)-3-phenyl-propan-1-one

61.0 g (0.17 mol) of 4-hydroxy-5-[1-((4R,5S)-2,2-dimethyl-5-phenyl-[1,3]dioxolan-4-yl)-methanoyl]-1,2-dimethyl-1H-benzoimidazole was dissolved in hydrochloric acid (155 ml) and stirred for 1 h at 25° C. Afterwards the reaction mixture was neutralized by adding sodium hydroxide solution (6N) and was extracted with dichloromethane/methanol (13/1) three times. The combined organic layers were concentrated in vacuo, reslurried from acetone and dried at 50° C. to give 42.7 g (0.13 mol/77%) of the title product as a colourless solid with a melting point of 170° C. (acetone).

### I. (7R,8R)-7-Acetox-2,3-dimethyl-8-phenyl-7,8-dihydro-3H-chromeno[7,8-d]imidazol-6-one

To a suspension of 29.7 g (91.0 mmol) (2R,3S)-2,3-dihydroxy-1-(4-hydroxy-1,2-dimethyl-1H-benzoimidazol-5-yl)-3-phenyl-propan-1-one in dichloromethane (300 ml) were added 47.6 ml (0.37 mol) trimethyl orthoacetate, 7.10 g (28.0 mmol) pyridinium p-toluenesulphonate and formic acid (15 ml) and it was stirred for 20 h at 25° C. The reaction was quenched by pouring out into water. Subsequently the mixture was neutralised by adding a sodium hydroxide solution and it was extracted with dichloromethane four times. The combined organic layers were concentrated in vacuo and purified by column chromatography (dichloromethane/methanol:100/3) to give 26.7 g (76.3 mmol/84%) of the title product as a colourless solid with a melting point of 248° C. (dichloromethane/methanol).

### J. (7R,8R)-7-Hydroxy-2,3-dimethyl-8-phenyl-7,8-dihydro-3H-chromeno[7,8-d]imidazol-6-one

41.6 g (0.13 mol) of (7R,8R)-7-acetoxy-2,3-dimethyl-8-phenyl-7,8-dihydro-3H-chromeno[7,8-d]imidazol-6-one in hydrochloric acid (2N/450 ml) was stirred for 5 days at 25° C. Subsequently the suspension was diluted with water and neutralized by adding a sodium hydroxide solution (6 N). The precipitate was filtered off, washed with water and dried at 50° C. under vacuum to give 37.0 g (0.12 mol/94%) of the title product as a beige solid with a melting point of 233° C. (water).

### K. (7R,8R)-2,3-Dimethyl-8-phenyl-7-pivaloxy-7,8-dihydro-3H-chromeno[7,8-]imidazol-6-one

To a at 0° C. cooled, stirred reaction mixture of 36.2 g (0.12 mol) (7R,8R)-7-hydroxy-2,3-dimethyl-8-phenyl-7,8-dihydro-3H-chromeno[7,8-d]imidazol-6-one, 40.9 ml (0.24 mol) ethyl diisoproylamine and 14.3 g (0.12 mol) 4-dimethylaminopyridine in dichloromethane (500 ml) were added slowly (over 45 min) 29.0 ml (0.24 mol) pivaloyl chloride and the reaction mixture was stirred for further 20 h at 0° C. The reaction mixture was quenched by pouring out into water and extracted with dichloromethane three times. The combined organic layers were concentrated in vacuo and purified by column chromatography (dichloromethane/methanol: 100/3) to give 45.9 g (0.12 mol/99%) of the title product as a colourless solid with a melting point of 255° C. (dichloromethane/methanol).

### L. 6R,7S,8R)-6-Hydroxy-2,3-dimethyl-8-phenyl-7-pivaloxy-3,6,7,8-tetrahydro-chromeno[7,8-d]imidazole

To a suspension of 10.0 g (25.5 mmol) (7R,8R)-2,3-dimethyl-8-phenyl-7-pivaloxy-7,8-dihydro-3H-chromeno[7,8-d]imidazol-6-one in methanol (200 ml) is added at −7° C. 1.00 g (26.4 mmol) sodium borohydride and is stirred for further 2 h at this temperature. Subsequently the reaction is quenched by adding saturated ammonium chloride solution. The precipitate is filtered off, washed with water and dried at 50° C. to give 9.98 g (25.4 mol/99%) of the title product as a colourless solid with a melting point of 148° C. (water).

### M. (6R,7S,8R)-(2-Fluoro-ethoxy)-2,3-dimethyl-8-phenyl-7-pivaloxy-3,6,7,8-tetrahydro-chromeno[7,8-d]imidazole

To a at −30° C. cooled suspension of 2.00 g (5.07 mmol) (6R,7S,8R)-6-hydroxy-2,3-dimethyl-8-phenyl-7-pivaloxy-3,6,7,8-tetrahydro-chromeno[7,8-d]imidazole in THF (20 ml) is added 6.40 ml (6.40 mmol) 2-fluoroethyl triflate (1M in dichloromethane) and 10.0 ml (10.0 mmol) bis-(trimethylsilyl)-sodium amide (1 M in THF). This mixture is stirred for 1 h at −30° C. Subsequently the reaction is quenched by pouring out into saturated ammonium chloride solution and it is extracted with dichloromethane three times. The combined organic layers are concentrated in vacuo and purified by column chromatography (dichloromethane/methanol:100/3) and reslurried from diethyl ether to give 0.80 g (1.82 mmol/36%) of the title product as a colourless solid with a melting point of 205° C. (diethyl ether).

### N. (6R,7S,8R)-6-Ethoxy-2,3-dimethyl-8-phenyl-7-pivaloxy-3,6,7,8-tetrahydro-chromeno[7,8-d]imidazole

To a at −78° C. cooled suspension of 5.00 g (12.7 mmol) (6R,7S,8R)-6-hydroxy-2,3-dimethyl-8-phenyl-7-pivaloxy-3,6,7,8-tetrahydro-3H-chromeno[7,8-d]imidazole in THF (100 ml) is added 2.45 g (13.8 mmol) ethyl triflate and 25.0 ml (25.0 mmol) bis-(trimethylsilyl)-sodium amide (1 M in THF). This mixture is stirred for 1 h at −30° C. Subsequently the reaction is quenched by pouring out into saturated ammonium chloride solution and is extracted with dichloromethane three times. The combined organic layers are concentrated in vacuo and purified by column chromatography (dichloromethane/methanol: 100/3) and reslurried from diethyl ether to give 2.51 g (5.94 mmol/47%) of the title product as a colourless solid with a melting point of 164° C. (dichloromethane/methanol).

### O. 2-(1-Cyclopropyl-methanoylamino)-3-hydroxy-cyclohex-2-enone

To a suspension of 8.00 g (41.0 mmol) 2-(1-cyclopropyl-methanoylamino)-resorcinol is added 1.80 g (45.0 mmol) and 4.30 g Raney nickel (type B113W/Degussa) and the reaction mixture is stirred in a circulation hydrogenation at 55° C. for 24 h. Subsequently the mixture is acidified to pH 3 by adding hydrochloric acid (2 N). The catalyst is filtered off and the filtrate is extracted with dichloromethane/methanol (13/1) ten times. The combined organic layers are concentrated in vacuo to give 6.34 g (31.8 mmol/79%) of the title product as a colourless solid with a melting point of 82° C. (dichloromethane/methanol).

### P. 2-Methyl-1,5,6,7-tetrahydro-benzoimidazol-4-one

To a mixture of 30.0 g (0.18 mol) 2-acetylamino-3-hydroxy-cyclohex-2-enone in toluene (300 ml) is added 42.0 g (0.55 mol) ammonium acetate and the reaction mixture is stirred under reflux for 6 d. Afterwards the mixture is concentrated in vacuo and the crude product is purified by column chromatography (dichlormethane/methanol:100/1) and crystallized from diethyl ether to yield 12.0 g (0.80 mol/45%) of the title product with a melting point of 229° C. (diethyl ether).

### Q. 1-Cyclopropyl-2-methyl-1,5,6,7-tetrahydro-benzoimidazol-4-one

To a mixture of 5.00 g (29.6 mmol) 2-acetylamino-3-hydroxy-cyclohex-2-enone in toluene (42 ml) is added acetic acid (5 ml) and 1.76 g (31.0 mmol) cyclopropylamine and the reaction mixture is stirred under reflux for 1.5 d. Subsequently the mixture is acidified to pH 3 by adding hydrochloric acid (2 N) and is stirred for further 1 h. Afterwards the mixture is neutralized by adding saturated hydrogen carbonate solution and concentrated in vacuo. The crude product is purified by column chromatography (dichlormethane/methanol:100/1) and crystallized from diethyl ether to yield 1.87 g (9.82 mmol/33%) of the title product with a melting point of 132° C. (diethyl ether).

### R. 1-Allyl-2-methyl-1,5,6,7-tetrahydro-benzoimidazol-4-one

To a mixture of 32.2 g (0.19 mol) 2-acetylamino-3-hydroxy-cyclohex-2-enone in toluene (270 ml) is added acetic acid (32 ml) and 13.0 g (0.23 mol) allylamine and the reaction mixture is stirred under reflux for 1.5 h. Subsequently the mixture is acidified to pH 3 by adding hydrochloric acid (2 N) and is stirred for further 1 h. Afterwards the mixture is neutralized by adding saturated hydrogen carbonate solution and concentrated in vacuo. The crude product is purified by column chromatography (ethyl acetate: 100) and crystallized from diethyl ether to yield 15.2 g (0.80 mol/42%) of the title product with a melting point of 78° C. (diethyl ether).

### S. 2-Methyl-4-oxo-4,5,6,7-tetrahydro-benzoimidazole-1-carboxylic acid tert-butyl ester

To a mixture of 5.00 g (33.0 mmol) 2-methyl-1,5,6,7-tetrahydro-benzoimidazol-4-one in dioxane (50 ml) is added 18.0 g (83.0 mmol) di-tert-butyl-dicarbonate and the mixture is stirred for further 18 h at 70° C. Subsequently the reaction is concentrated in vacuo and the crude product is purified by column chromatography (dichloromethane/methanol:100/3) and crystallized from diethyl ether to yield 1.09 g (4.35 mol/13%) of the title product with a melting point of 126° C. (diethyl ether).

### T. 1-Benzyl-2-methyl-1,5,6,7-tetrahydro-benzoimidazol-4-one

To a mixture of 5.00 g (29.6 mmol) 2-acetylamino-3-hydroxy-cyclohex-2-enone in xylene (40 ml) is added acetic acid (5 ml) and 3.75 g (35.0 mmol) benzylamine and the reaction mixture is stirred under reflux for 2 h. Subsequently the mixture is acidified to pH 3 by adding hydrochloric acid (2 N) and is stirred for further 1 h. Afterwards the mixture is neutralized by adding saturated hydrogen carbonate solution and concentrated in vacuo. The crude product is purified by column chromatography (dichloromethane/methanol:100/3), (ethyl acetate: 100) and crystallized from diethyl ether to yield 4.22 g (17.6 mol/59%) of the title product with a melting point of 115° C. (diethyl ether).

### U. 2-Cyclopropyl-1-methyl-1,5,6,7-tetrahydro-benzoimidazol-4-one

To a mixture of 13.0 g (66.7 mmol) 2-(1-cyclopropyl-methanoylamino)-3-hydroxy-cyclohex-2-enone in xylene (260 ml) is added acetic acid (13 ml) and methylamine (26 ml). The reaction mixture is stirred under reflux for 17 h. Subsequently the mixture is acidified to pH 3 by adding hydrochloric acid (2 N) and is stirred for further 1 h. Afterwards the mixture is neutralized by adding saturated hydrogen carbonate solution and concentrated in vacuo. The crude product is purified by column chromatography (dichlormethane/methanol:100/1) and crystallized from diethyl ether to yield 8.50 g (44.7 mmol/67%) of the title product with a melting point of 108° C. (diethyl ether).

### V. 1-Allyl-5-[1-((4R,5S)-2,2-dimethyl-5-phenyl[1,3]dioxolan-4-yl)methanoyl]-2-methyl-1,5,6,7-tetrahydro-benzoimidazol-4-one

To a solution of 2.00 g (10.5 mmol) of freshly oven dried 1-allyl-2-methyl-1,5,6,7-tetrahydro-benzoimidazol-4-one in THF (50 ml) is added 0.76 g (19.0 mmol) sodium hydride (60% dispersion in mineral oil) and is stirred for 0.5 h at 25° C. and for 1 h at 60° C.

At the same time acetonide protected (2R,3S)-2,3-dihydroxy-3-phenylpropoinic acid is activated by following procedure.

To a stirred solution of 5.14 g (23.1 mmol) acetonide protected (2R,3S)-2,3-dihydroxy-3-phenylpropoinic acid in THF (10 ml) is added portion wise 4.10 g (25.3 mmol) N,N′-carbonyldiimidazole. This solution is stirred for 2 h at 25° C.

Afterwards this solution is added dropwise to the at 60° C. stirred reaction suspension and is stirred for further 1 h. Subsequently the reaction is quenched by pouring out into a saturated ammonium chloride solution. The organic layer is separated and the water layer is extracted with dichloromethane two times. The combined organic layers are concentrated in vacuo and purified by column chromatography (dichloromethane/methanol:100/1) and (ethyl acetate: 100) to give 1.67 g (4.23 mmol/40%) of the title product as a colourless solid with a melting point of 117° C. (ethyl acetate).

### W. 1-Cyclopropyl-5-[1-(4R,5S)-2,2-dimethyl-5-phenyl[1,3]dioxolan-4-yl)methanoyl]-2-methyl-1,5,6,7-tetrahydro-benzoimidazol-4-one

To a solution of 1.00 g (5.26 mmol) of freshly oven dried 1-cyclopropyl-2-methyl-1,5,6,7-tetrahydro-benzoimidazol-4-one in THF (25 ml) is added 0.40 g (10.0 mmol) sodium hydride (60% dispersion in mineral oil) and is stirred for 0.5 h at 25° C. and for 1 h at 60° C.

At the same time acetonide protected (2R,3S)-2,3-dihydroxy-3-phenylpropoinic acid is activated by following procedure.

To a stirred solution of 2.60 g (11.7 mmol) acetonide protected (2R,3S)-2,3-dihydroxy-3-phenylpropoinic acid in THF (10 ml) is added portion wise 2.00 g (12.3 mmol) N,N′-carbonyldiimidazole. This solution is stirred for 2 h at 25° C.

Afterwards this solution is added dropwise to the at 60° C. stirred reaction suspension and is stirred for further 1.5 h. Subsequently the reaction is quenched by pouring out into a saturated ammonium chloride solution. The organic layer is separated and the water layer is extracted with dichloromethane three times. The combined organic layers are concentrated in vacuo and purified by column chromatography (dichloromethane/methanol:100/1) to give 1.16 g (2.94 mmol/56%) of the title product as a colourless oil. This product is used in the next reaction step without further purification and characterisation.

### X. 2-Cyclopropyl-5-[1-((4R,5S)-2,2-dimethyl-5-phenyl[1,3]dioxolan-4-yl)methanoyl]-1-methyl-1,5,6,7-tetrahydro-benzoimidazol-4-one

To a solution of 8.40 g (44.0 mmol) of freshly oven dried 2-cyclopropyl-1-methyl-1,5,6,7-tetrahydro-benzoimidazol-4-one in THF (217 ml) is added 3.40 g (85.0 mmol) sodium hydride (60% dispersion in mineral oil) and is stirred for 0.5 h at 25° C. and for 1 h at 60° C.

At the same time acetonide protected (2R,3S)-2,3-dihydroxy-3-phenylpropoinic acid is activated by following procedure.

To a stirred solution of 22.4 g (101 mmol) acetonide protected (2R,3S)-2,3-dihydroxy-3-phenylpropoinic acid in THF (10 ml) is added portion wise 16.8 g (104 mmol) N,N′-carbonyldiimidazole. This solution is stirred for 2 h at 25° C.

Afterwards this solution is added dropwise to the at 60° C. stirred reaction suspension and is stirred for further 0.5 h. Subsequently the reaction is quenched by pouring out into a saturated ammonium chloride solution. The organic layer is separated and the water layer is extracted with dichloromethane three times. The combined organic layers are concentrated in vacuo and purified by column chromatography (dichloromethane/methanol:100/1) to give 12.3 g (31.2 mmol/70%) of the title product as a colourless oil. This product is used in the next reaction step without further purification and characterisation.

### Y. 1-Allyl-4-hydroxy-5-[1-((4R,5S)-2,2-dimethyl-5-phenyl-[1,3]dioxolan-4-yl)-methanoyl]-2-methyl-1H-benzoimidazole

31.0 g (78.6 mmol) 1-allyl-5-[1-((4R,5S)-2,2-dimethyl-5-phenyl[1,3]dioxolan-4-yl)methanoyl]-2-methyl-1,5,6,7-tetrahydro-benzoimidazol-4-one and 67.3 g (77.5 mmol) manganese(IV)oxide in dioxan (634 ml) are stirred for 24 h at 25° C. Afterwards the reaction mixture is filtered over sand/kieselgur and the filtrate is concentrated in vacuo. The crude product is crystallized from diethyl ether to give 25.8 g (65.7 mmol/84%) of the title product as a colourless solid with a melting point of 123° C. (diethyl ether).

### Z. 4-Hydroxy-5-[1-((4R,5S)-2,2-dimethyl-5-phenyl-[1,3]dioxolan-4-yl)-methanoyl]-2-methyl-1-propenyl-1H-benzoimidazole*1

30.0 g (76.0 mmol) 5-[1-((4R,5S)-2,2-dimethyl-5-phenyl[1,3]dioxolan-4-yl)methanoyl]-2-methyl-1-propenyl-1,5,6,7-tetrahydro-benzoimidazol-4-one and 65.2 g (75.0 mmol) manganese(IV)oxide in dioxan (614 ml) are stirred for 24 h at 25° C. Afterwards the reaction mixture is filtered over sand/kieselgur and the filtrate is concentrated in vacuo. The crude product is purified by column chromatography (dichloromethane/methanol:100/1) and crystallized from diisopropyl ether to give 9.24 g (23.5 mmol/31%) of the title product as a colourless solid with a melting point of 122° C.*1 (diisopropyl ether).

*1=The product is a mixture of E/Z-isomeres (2/1) in 1-propenyl.

### AA. 1-Cyclopropyl-4-hydroxy-5-[1-((4R,5S)-2,2-dimethyl-5-phenyl-[1,3]dioxolan-4-yl)-methanoyl]-2-methyl-1H-benzoimidazole

1.00 g (2.53 mmol) 1-cyclopropyl-5-[1-((4R,5S)-2,2-dimethyl-5-phenyl[1,3]dioxolan-4-yl)methanoyl]-2-methyl-1,5,6,7-tetrahydro-benzoimidazol-4-one and 2.20 g (25.2 mmol) manganese(IV)oxide in dioxan (20 ml) are stirred for 5 h at 25° C. Afterwards the reaction mixture is filtered over sand/kieselgur and the filtrate is concentrated in vacuo. The crude product is purified by column chromatography (dichloromethane/methanol:100/1) to give 0.84 g (2.14 mmol/84%) of the title product as a colourless solid with a melting point of 153° C. (dichloromethane/methanol).

### BB. 2-Cyclopropyl-4-hydroxy-5-[1-((4R,5S)-2,2-dimethyl-5-phenyl-[1,3]dioxolan-4-yl)-methanoyl]-1-methyl-1H-benzoimidazole

250 mg (0.63 mmol) 2-cyclopropyl-5-[1-((4R,5S)-2,2-dimethyl-5-phenyl[1,3]dioxolan-4-yl)methanoyl]-1-methyl-1,5,6,7-tetrahydro-benzoimidazol-4-one and 550 mg (6.33 mmol) manganese(IV)oxide in dioxan (5 ml) are stirred for 24 h at 25° C. Afterwards the reaction mixture is filtered over sand/kieselgur and the filtrate is concentrated in vacuo. The crude product is purified by column chromatography (dichloromethane/methanol:100/3) to give 260 mg (0.66 mmol/87%) of the title product as a oil.

1H-NMR (200 MHz, CDCl3): δ=1.05-1.14 (m, 2H), 1.22-1.35 (m, 2H), 1.57 (s, 3H), 1.70 (s, 3H), 1.86-2.05 (m, 1H), 3.78 (s, 3H), 5.11 (d, 1H), 5.46 (d, 1H), 6.60 (d, 1H), 7.27-7.53 (m, 6H).

### CC. (2R,3S)-2,3-Dihydroxy-1-(1-allyl-4-hydroxy-2-methyl-1H-benzoimidazol-5-yl)-3-phenyl-propan-1-one

10.8 g (27.5 mmol) of 1-allyl-4-hydroxy-5-[1-((4R,5S)-2,2-dimethyl-5-phenyl-[1,3]dioxolan-4-yl)-methanoyl]-2-methyl-1H-benzoimidazole is dissolved in hydrochloric acid (6N) (32 ml) and stirred for 1 h at 25° C. Afterwards the reaction mixture is neutralized by adding sodium hydroxide solution (6N) and is extracted with dichloromethane three times. The combined organic layers are concentrated in vacuo, crystallized from diisopropyl ether and dried at 50° C. to give 9.64 g (26.8 mmol/99%) of the title product as a colourless solid with a melting point of 108° C. (diisopropyl ether).

### DD. (2R,3S)-2,3-Dihydroxy-1-(4-hydroxy-2-methyl-1-propenyl-1H-benzoimidazol-5-yl)-3-phenyl-propan-1-one*1

22.0 g (56.0 mmol) of 4-hydroxy-5-[1-((4R,5S)-2,2-dimethyl-5-phenyl-[1,3]dioxolan-4-yl)-methanoyl]-2-methyl-1-propenyl-1H-benzoimidazole is dissolved in hydrochloric acid (6N) (66 ml) and stirred for 4 h at 25° C. Afterwards the reaction mixture is neutralized by adding sodium hydroxide solution (6N) and is extracted with dichloromethane three times. The combined organic layers are concentrated in vacuo, crystallized from diisopropyl ether and dried at 50° C. to give 16.5 g (46.8 mmol/84%) of the title product as a colourless solid with a melting point of 103° C.*1 (diisopropyl ether).

*1=The product is a mixture of E/Z-isomeres (2/1) in 1-propenyl.

### EE. (2R,3S)-2,3-Dihydroxy-1-(1-cyclopropyl-4-hydroxy-2-methyl-1H-benzoimidazol-5-yl)-3-phenyl-propan-1-one

29.0 g (70.0 mmol) of 1-cyclopropyl-4-hydroxy-5-[1-((4R,5S)-2,2-dimethyl-5-phenyl-[1,3]dioxolan-4-yl)-methanoyl]-2-methyl-1H-benzoimidazole is dissolved in hydrochloric acid (6N) (180 ml) and stirred for 0.5 h at 25° C. Afterwards the reaction mixture is neutralized by adding sodium hydroxide solution (6N) and is extracted with dichloromethane three times. The combined organic layers are concentrated in vacuo, crystallized from diethyl ether and dried at 60° C. to give 21.2 g (60.2 mmol/82%) of the title product as a colourless solid with a melting point of 120° C. (diethyl ether).

### FF. (2R,3S)-2,3-Dihydroxy-1-(2-cyclopropyl-4-hydroxy-1-methyl-1H-benzoimidazol-5-yl)-3-phenyl-propan-1-one

6.20 g (15.8 mmol) of 2-cyclopropyl-4-hydroxy-5-[1-((4R,5S)-2,2-dimethyl-5-phenyl-[1,3]dioxolan-4-yl)-methanoyl]-1-methyl-1H-benzoimidazole is dissolved in hydrochloric acid (6N) (40 ml) and stirred for 50 min at 25° C. Afterwards the reaction mixture is pouring out into a sodium hydroxide solution (6N), neutralized by adding saturated sodium hydrogen carbonate solution and extracted with dichloromethane three times. The combined organic layers are concentrated in vacuo and dried at 60° C. to give 4.84 g (13.7 mmol/87%) of the title product as a colourless solid with a melting point of 156° C. (dichloromethane).

### GG. (7R,8R)-7-Acetoxy-2-methyl-8-phenyl-7,8-dihydro-3H-chromeno[7,8-d]imidazol-6-ne

To a stirred reaction mixture of 1.00 g (2.66 mmol) (7R,8R)-7-acetoxy-2-methyl-8-phenyl-3-propenyl-7,8-dihydro-3H-chromeno[7,8-d]imidazol-6-one in acetone (10 ml) is added 1.68 g (10.6 mmol) potassium permanganate and it is stirred for 5 h at 25° C. The reaction is quenched by adding saturated sodium hydrogen sulphite solution. After filtration the filtrate is extracted with dichloromethane two times and the combined organic layers are concentrated in vacuo to give 0.52 g (1.55 mmol/58%) of the product as light brown foam.

1H-NMR (200 MHz, CDCl3): δ=2.00 (s, 3H), 2.57 (s, 3H), 5.53 (d, 1H), 5.90 (d, 1H), 7.31-7.48 (m, 6H), 7.76 (d, 1H).

### HH. (7R,8R)-7-Acetoxy-3-allyl-2-methyl-8-phenyl-7,8-dihydro-3H-chromeno[7,8-d]imidazol-6-one

To a suspension of 1.00 g (2.84 mmol) (2R,3S)-2,3-dihydroxy-1-(4-hydroxy-1-allyl-2-methyl-1H-benzoimidazol-5-yl)-3-phenyl-propan-1-one in dichloromethane (10 ml) is added 1.60 ml (12.4 mmol) trimethyl orthoacetate, 0.24 g (0.95 mmol) pyridinium p-toluenesulphonate and formic acid (0.5 ml) and it is stirred for 2 h at 25° C.

The reaction is quenched by pouring out into a saturated sodium carbonate solution and is extracted with dichloromethane three times. The combined organic layers are concentrated in vacuo and purified by column chromatography (dichloromethane/methanol:100/3) to give 0.64 g (1.70 mmol/60%) of the title product with a melting point of 113° C. (dichloromethane/methanol).

### II. (7R,8R)-7-Acetoxy-2-methyl-8-phenyl-3-propenyl-7,8-dihydro-3H-chromeno[7,8-d]imidazol-6-one*1

To a suspension of 16.0 g (45.5 mmol) (2R,3S)-2,3-dihydroxy-1-(4-hydroxy-1-propenyl-2-methyl-1H-benzoimidazol-5-yl)-3-phenyl-propan-1-one in dichloro-methane (160 ml) is added 25.7 ml (0.20 mol) trimethyl orthoacetate, 5.30 g (15.8 mmol) pyridinium p-toluenesulphonate and formic acid (8 ml) and it is stirred for 5 h at 25° C.

The reaction is quenched by pouring out into a saturated sodium carbonate solution and is extracted with dichloromethane three times. The combined organic layers are concentrated in vacuo and purified by column chromatography (dichloromethane/methanol: 100/3) and the product is crystallized from diisopropyl ether to give 7.36 g (19.5 mmol/43%) of the title compound with a melting point of 189° C.*1 (diisopropyl ether).

*1=The product is a mixture of E/Z-isomeres (3/1) in 3-propenyl.

### JJ. (7R,8R)-7-Acetoxy-3-cyclopropyl-2-methyl-8-phenyl-7,8-dihydro-3H-chromeno[7,8-d]imidazol-6-one

To a suspension of 21.2 g (60.0 mmol) (2R,3S)-2,3-dihydroxy-1-(4-hydroxy-1-cyclopropyl-2-methyl-1H-benzoimidazol-5-yl)-3-phenyl-propan-1-one in dichloromethane (265 ml) is added 34.0 ml (0.26 mol) trimethyl orthoacetate, 5.30 g (21.0 mmol) pyridinium p-toluenesulphonate and formic acid (42 ml) and it is stirred for 2 h at 25° C.

The reaction is quenched by pouring out into a saturated sodium carbonate solution and is extracted with dichloromethane three times. The combined organic layers are concentrated in vacuo and purified by column chromatography (dichloromethane/methanol:100/3) to give 15.5 g (41.2 mmol/69%) of the title product with a melting point of 250° C. (dichloromethane/methanol).

### KK. (7R,8R)-Allyl-2-methyl-8-phenyl-7-pivaloxy-7,8-dihydro-3H-chromeno[7,8-d]imidazol-one

To a at 0° C. cooled stirred reaction mixture of 1.40 g (4.19 mmol) (7R,8R)-3-allyl-7-hydroxy-2-methyl-8-phenyl-7,8-dihydro-3H-chromeno[7,8-d]imidazol-6-one, 1.40 ml (8.04 mmol) ethyl diisoproylamine and 0.50 g (4.09 mmol) 4-dimethylaminopyridine in dichloromethane (19 ml) is added slowly 1.0 ml (8.12 mmol) pivaloyl chloride and is stirred for further 26 h at 0° C. The reaction is quenched by pouring out into ice water and is extracted with dichloromethane three times. The combined organic layers are concentrated in vacuo and purified by column chromatography (dichloromethane/methanol:100/3) and the product is recrystallized from diethyl ether to give 1.28 g (3.06 mmol/73%) of the title compound as a colourless-solid with a melting point of 151° C. (diethyl ether).

### LL. (7R,8R)-2-Methyl-8-phenyl-7-pivaloxy-3-propenyl-7,8-dihydro-3H-chromeno[7,8-d]imidazol-6-one*1

To a at 0° C. cooled stirred reaction mixture of 8.00 g (24.0 mmol) (7R,8R)-7-hydroxy-2-methyl-8-phenyl-3-propenyl-7,8-dihydro-3H-chromeno[7,8-d]imidazol-6-one, 8.00 ml (46.0 mmol) ethyl diisoproylamine and 2.80 g (24.0 mmol) 4-dimethylaminopyridine in dichloromethane (109 ml) is added slowly 5.70 ml (46.0 mmol) pivaloyl chloride and is stirred for further 22 h at 0° C. The reaction is quenched by pouring out into ice water and is extracted with dichloromethane three times. The combined organic layers are concentrated in vacuo and purified by column chromatography (dichloromethane/methanol:100/1) and the product is recrystallized from diethyl ether to give 3.57 g (8.53 mmol/40%) of the title compound as a colourless solid with a melting point of 213° C.*1 (diethyl ether).

*1=The product is a mixture of E/Z-isomeres (1/1) in 3-propenyl.

### MM. (7R,8R)-7-Acetoxy-2-cyclopropyl-3-methyl-8-phenyl-7,8-dihydro-3H-chromeno[7,8-d]imidazol-6-one

To a suspension of 4.20 g (60.0 mmol) (2R,3S)-2,3-dihydroxy-1-(4-hydroxy-2-cyclopropyl-1-methyl-1H-benzoimidazol-5-yl)-3-phenyl-propan-1-one in dichloro-methane (53 ml) is added 6.70 ml (51.0 mmol) trimethyl orthoacetate, 1.10 g (4.36 mmol) pyridinium p-toluenesulphonate and formic acid (8 ml) and it is stirred for 1 h at 25° C.

The reaction is quenched by pouring out into a saturated sodium carbonate solution and is extracted with dichloromethane three times. The combined organic layers are concentrated in vacuo and purified by column chromatography (dichloromethane/methanol:100/3) to give 3.40 g (9.03 mmol/76%) of the title product with a melting point of 205° C. (dichloromethane/methanol).

### NN. (7R,8R)-3-Allyl-7-hydroxy-2-methyl-8-phenyl-7,8-dihydro-3H-chromeno[7,8-d]imidazol-one

17.5 g (46.5 mmol) of (7R,8R)-3-allyl-7-acetoxy-2-methyl-8-phenyl-7,8-dihydro-3H-chromeno[7,8-d]imidazol-6-one in hydrochloric acid (2N/175 ml) is stirred for 5 h at 50° C. Subsequently the suspension is diluted with water and neutralized by adding sodium hydroxide solution (6 N). The precipitate is filtered off, washed with water and dried by 50° C. under vacuum to give 15.0 g (44.9 mmol/97%) of the title product as a colourless solid with a melting point of 169° C. (water).

### OO. (7R,8R)-7-Hydroxy-2-methyl-8-phenyl-3-propenyl-7,8-dihydro-3H-chromeno[7,8-d]imidazol-6-ne

9.00 g (24.0 mmol) of (7R,8R)-7-acetoxy-2-methyl-8-phenyl-3-propenyl-7,8-dihydro-3H-chromeno[7,8-d]imidazol-6-one in hydrochloric acid (2N/90 ml) is stirred for 24 h at 50° C. Subsequently the suspension is diluted with water and neutralized by adding sodium hydroxide solution (6 N). The precipitate is filtered off, washed with water and dried by 50° C. under vacuum to give 8.40 g of the title product as a colourless solid. This compound is used without further purification and characterisation in followed reactions.

### PP. (7R,8R)-3-Cyclopropyl-7-hydroxy-2-methyl-8-phenyl-7,8-dihydro-3H-chromeno[7,8-d]imidazol-6-one

15.0 g (40.0 mmol) of (7R,8R)-3-cyclopropyl-7-acetoxy-2-methyl-8-phenyl-7,8-dihydro-3H-chromeno[7,8-d]imidazol-6-one in hydrochloric acid (2N/150 ml) is stirred for 4 d at 50° C. Subsequently the suspension is diluted with water and neutralized by adding sodium hydroxide solution (6 N). The precipitate is filtered off, washed with water and dried by 50° C. under vacuum to give 12.1 g (36.2 mmol/92%) of the title product as a colourless solid with a melting point of 178° C. (water).

### QQ. (6R,7S,8R)-3-Allyl-6-(2-fluoro-ethoxy)-2-methyl-8-phenyl-7-pivaloxy-3,6,7,8-tetrahydro-chromeno[7,8-d]imidazole

To a at −30° C. cooled suspension of 6.70 g (15.9 mmol) (6R,7S,8R)-3-allyl-6-hydroxy-2-methyl-8-phenyl-7-pivaloxy-3,6,7,8-tetrahydro-chromeno[7,8-d]imidazole in THF (70 ml) is added 21.5 ml (21.5 mmol) 2-fluoroethyl triflate (1 M in dichloromethane) and 33.5 ml (33.5 mmol) bis-(trimethylsilyl)-sodium amide (1 M in THF). This mixture is stirred for 1 h at −30° C. Subsequently the reaction is quenched by pouring out into saturated ammonium chloride solution and is extracted with dichloromethane three times. The combined organic layers are concentrated in vacuo and purified by column chromatography (dichloromethane/methanol: 100/3) to give 6.83 g (14.6 mmol/92%) of the title product as a colourless foam.

1H-NMR (200 MHz, CDCl3): δ=0.95 (s, 9H), 2.55 (s, 3H), 3.56-3.90 (m, 2H), 4.33 (t, 1H), 4.58 (t, 1H), 4.68-4.72 (m, 2H), 4.95 (d, 1H), 5.01-5.30 (m, 3H), 5.73-6.03 (m, 2H), 6.95 (d, 1H), 7.26-7.37 (m, 3H), 7.46-7.51 (m, 2H).

RR. (6R,7S,8R)-3-Allyl-6-hydroxy-2-methyl-8-phenyl-7-pivaloxy-3,6,7,8-tetrahydro-chromeno[7,8-d]imidazole

To a suspension of 7.50 g (17.9 mmol) (7R,8R)-3-allyl-2-methyl-8-phenyl-7-pivaloxy-7,8-dihydro-3H-chromeno[7,8-d]imidazol-6-one in methanol (130 ml) is added at −7° C. 0.75 g (19.8 mmol) sodium borohydride and is stirred for further 2 h at this temperature. Subsequently the reaction is quenched by adding saturated ammonium chloride solution. The precipitate is filtered off, washed with water and dried at 60° C. to give 7.00 g (16.7 mol/93%) of the title product as a colourless solid with a melting point of 119° C. (water).

### SS. (6R,7S,8R)-6-Hydroxy-2-methyl-8-phenyl-7-pivaloxy-3-propenyl-3,6,7,8-tetrahydro-chromeno[7,8-d]imidazole*1

To a suspension of 5.00 g (12.0 mmol) (7R,8R)-2-methyl-8-phenyl-7-pivaloxy-3-propenyl-7,8-dihydro-3H-chromeno[7,8-d]imidazol-6-one in methanol (90 ml) is added at −7° C. 0.50 g (13.2 mmol) sodium borohydride and is stirred for further 1 h at this temperature. Subsequently the reaction is quenched by adding saturated ammonium chloride solution. The precipitate is filtered off, washed with water and dried at 60° C. to give 4.61 g (11.0 mol/92%) of the title product as a colourless solid with a melting point of 123° C.*1 (water).

*1=The product is a mixture of E/Z-isomeres (4/1) in 3-propenyl.

### TT. (6R,7S,8R)-2-Methyl-6-(2-methoxy-ethoxy)-8-phenyl-7-pivaloxy-3-propenyl-3,6,7,8-tetrahydro-chromeno[7,8-d]imidazole

To a at −40° C. cooled suspension of 4.30 g (10.2 mmol) (6R,7S,8R)-6-hydroxy-2-methyl-8-phenyl-7-pivaloxy-3-propenyl-3,6,7,8-tetrahydro-chromeno[7,8-d]imidazole in THF (86 ml) is added 2.20 g (10.6 mmol) 2-methoxyethyl triflate and 23.8 ml (23.8 mmol) bis-(trimethylsilyl)-sodium amide (1 M in THF). This mixture is stirred for 1 h at −30° C. Subsequently the reaction is quenched by pouring out into saturated ammonium chloride solution and it is extracted with dichloromethane three times. The combined organic layers are concentrated in vacuo and purified by column chromatography (dichloromethane/methanol:100/3) to give 1.10 g (2.30 mmol/22%) of the title product as a colourless oil.

This compound is used without further purification and characterisation in followed reactions.

### UU. (6R,7S,8R)-3-Allyl-2-methyl-6-(2-methoxy-ethoxy)-8-phenyl-7-pivaloxy-3,6,7,8-tetrahydro-chromeno[7,8-d]imidazole

To a at −40° C. cooled suspension of 4.60 g (11.0 mmol) (6R,7S,8R)-3-allyl-6-hydroxy-2-methyl-8-phenyl-7-pivaloxy-3,6,7,8-tetrahydro-chromeno[7,8-d]imidazole in THF (92 ml) is added 2.30 g (11.0 mmol) 2-methoxyethyl triflate and 23.0 ml (23.0 mmol) bis-(trimethylsilyl)-sodium amide (1 M in THF). This mixture is stirred for 1 h at −40° C. Subsequently the reaction is quenched by pouring out into saturated ammonium chloride solution and it is extracted with dichloromethane three times. The combined organic layers are concentrated in vacuo and purified by column chromatography (dichloromethane/methanol:100/1) to give 3.91 g (8.12 mmol/75%) of the title product as a colourless oil.

1H-NMR (200 MHz, CDCl3): δ=0.95 (s, 9H), 2.54 (s, 3H), 3.34 (s, 3H), 3.44-3.75 (m, 4H), 4.67-4.71 (m, 2H), 4.94 (d, 1H), 5.08-5.24 (m, 3H), 5.75-6.03 (m, 2H), 6.93 (d, 1H), 7.27-7.37 (m, 4H), 7.46-7.51 (m, 2H).

### VV. (6R,7S,8R)-2-Methyl-6-(2-methoxy-ethoxy)-8-phenyl-7-pivaloxy-3,6,7,8-tetrahydro-chromeno[7,8-d]imidazole

To a stirred solution of 0.85 g (1.78 mmol) (6R,7S,8R)-2-methyl-6-(2-methoxy-ethoxy)-8-phenyl-7-pivaloxy-3-propenyl-3,6,7,8-tetrahydro-chromeno[7,8-d]imidazole in acetone (10 ml) is added 0.98 g (7.08 mmol) potassium permanganate and it is stirred for further 2 h. The reaction is quenched by adding saturated sodium hydrogen sulphite solution. Afterwards the mixture is filtered off and the filtrate is extracted with dichloromethane three times.

The combined organic layers are concentrated in vacuo and purified by column chromatography (dichloromethane/methanol: 100/1) to give 0.50 g (1.14 mmol/64%) of the title product as a colourless solid with a melting point of 110° C. (dichloromethane/methanol).

### WW. (6R,7S,8R)-2,3-dimethyl-6-(2-methoxy-ethoxy)-8-phenyl-7-pivaloxy-3,6,7,8-tetrahydro-chromeno[7,8-d]imidazole

To a at −40° C. cooled suspension of 2.00 g (5.07 mmol) (6R,7S,8R)-6-hydroxy-2,3-dimethyl-8-phenyl-7-pivaloxy-3,6,7,8-tetrahydro-chromeno[7,8-d]imidazole in THF (40 ml) is added 1.08 g (5.17 mmol) 2-methoxyethyl triflate and 10.4 ml (10.4 mmol) bis-(trimethylsilyl)-sodium amide (1 M in THF). This mixture is stirred for 1 h at −40° C. Subsequently the reaction is quenched by pouring out into saturated ammonium chloride solution and it is extracted with dichloromethane three times. The combined organic layers are concentrated in vacuo and purified by column chromatography (dichloromethane/methanol: 95/5) to give 1.50 g (3.31 mmol/65%) of the title product as a pale yellow foam. This product is transformed without further purification and characterisation under standard basic condition with potassium carbonate in methanol into compound 1.

### INDUSTRIAL APPLICABILITY

The compounds of the formulae 1 and 1a and their pharmacologically acceptable salts (=active compounds according to the invention) have valuable pharmacological properties which make them commercially utilizable. In particular, they exhibit marked inhibition of gastric acid secretion and an excellent gastric and intestinal protective action in warm-blooded animals, in particular humans. In this connection, the active compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the active compounds according to the invention surprisingly prove to be clearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the abovementioned diseases.

The invention furthermore includes the use of the active compounds according to the invention for the treatment and/or prophylaxis of the abovementioned diseases.

A further subject of the invention are medicaments which comprise one or more active compounds according to the invention.

The medicaments are prepared by processes which are known per se and familiar to the person skilled in the art. As medicaments, the active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the active compounds according to the invention and/or their salts are to be used for the treatment of the abovementioned diseases, the pharmaceutical preparations can also contain one or more pharmacologically active constituents of other groups of medicaments, for example: tranquilizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the active compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, meziocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the active compounds according to the invention are suited for a free or fixed combination with those medicaments (e.g. certain antiinflammatories and antirheumatics, such as NSAIDs), which are known to have a certain ulcerogenic potency. In addition, the active compounds according to the invention are suited for a free or fixed combination with motility-modifying drugs.

1. A compound of the formula 1 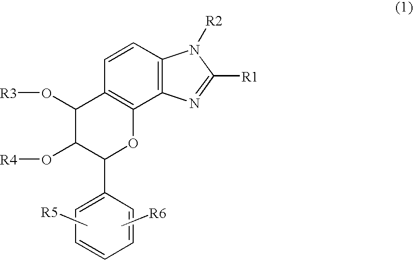
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form a methylene (—CH₂—), an ethylene (—CH₂—CH₂—), a propylene (—CH₂—CH₂—CH₂—) or a isopropylidene (—C(CH₃)₂—) radical,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof.

2. A compound of the formula 1 as claimed in claim 1, in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
or where R3 and R4 together form a methylene (—CH₂—), an ethylene (—CH₂—CH₂—), a propylene (—CH₂—CH₂—CH₂—) or a isopropylidene (—C(CH₃)₂—) radical,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof.

3. A compound of the formula 1 as claimed in claim 1, in which
R1 is hydrogen, 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form a methylene (—CH₂—), an ethylene (—CH₂—CH₂—), a propylene (—CH₂—CH₂—CH₂—) or a isopropylidene (—C(CH₃)₂—) radical,
R5 is hydrogen, fluoro, 1-4C-alkyl or fluoro-1-4C-alkyl,
R6 is hydrogen, fluoro, 1-4C-alkyl or fluoro-1-4C-alkyl,
or a salt thereof.

4. A compound of the formula 1 as claimed in claim 1, in which
R1 is hydrogen, 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form an ethylene (—CH₂—CH₂—) radical,
R5 is hydrogen and
R6 is hydrogen,
or a salt thereof.

5. A compound of the formula 1 as claimed in claim 1, in which
R1 is 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 1-4C-alkoxy-1-4C-alkyl or fluoro-1-4C-alkyl,
R4 is hydrogen, 1-4C-alkoxy-1-4C-alkylcarbonyl or mono- or di-1-4C-alkylamino-1-4C-alkylcarbonyl, or where R3 and R4 together form an ethylene (—CH₂—CH₂—) radical,
R5 is hydrogen,
R6 is hydrogen,
or a salt thereof.

6. A compound of the formula 1 as claimed in claim 1, in which
R1 is 1-4C-alkyl,
R2 is 1-4C-alkyl,
R3 is hydrogen, 1-4C-alkyl, 1-4C-alkoxy-1-4C-alkyl or fluoro-1-4C-alkyl,
R4 is hydrogen,
or where R3 and R4 together form an ethylene (—CH2-CH2-) radical,
R5 is hydrogen,
R6 is hydrogen,
or a salt thereof.

7. A compound of the formula 1 as claimed in claim 1, characterized by the formula 1-a, 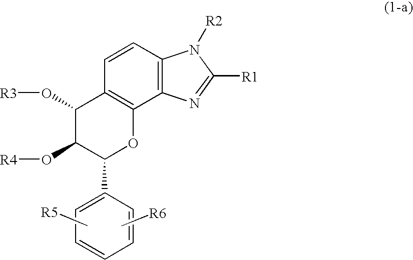
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,
or where R3 and R4 together form a methylene (—CH₂—), an ethylene (—CH₂—CH₂—), a propylene (—CH₂—CH₂—CH₂—) or a isopropylidene (—C(CH₃)₂—) radical,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof.

8. A compound of the formula 1-a as claimed in claim 7, in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
or where R3 and R4 together form a methylene (—CH₂—), an ethylene (—CH₂—CH₂—), a propylene (—CH₂—CH₂—CH₂—) or a isopropylidene (—C(CH₃)₂—) radical,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof.

9. A compound of the formula 1-a as claimed in claim 7, in which
R1 is 1-4C-alkyl or 3-7C-cycloalkyl,
R2 is hydrogen, 1-4C-alkyl, 3-7C-cycloalkyl or 2-4C-alkenyl,
R3 is hydrogen, 1-4C-alkyl, 1-4C-alkoxy-1-4C-alkyl or fluoro-1-4C-alkyl,
R4 is hydrogen, 1-4C-alkoxy-1-4C-alkylcarbonyl or mono- or di-1-4C-alkylamino-1-4C-alkylcarbonyl,
or where R3 and R4 together form an ethylene (—CH₂—CH₂—) radical,
R5 is hydrogen,
R6 is hydrogen,
or a salt thereof.

10. A compound of the formula 1-a as claimed in claim 7, in which
R1 is 1-4C-alkyl,
R2 is 1-4C-alkyl,
R3 is hydrogen, 1-4C-alkyl, 1-4C-alkoxy-1-4C-alkyl or fluoro-1-4C-alkyl,
R4 is hydrogen,
or where R3 and R4 together form an ethylene ethylen (—CH₂—CH₂—) radical,
R5 is hydrogen,
R6 is hydrogen,
or a salt thereof.

11. A pharmaceutical composition comprising a compound as claimed in claim 1 and/or a pharmacologically acceptable salt thereof together with a pharmaceutically acceptable auxiliary and/or excipient.

12. (canceled)

13. A method of preventing or treating a gastrointestinal disorder in a patient comprising administering to a patient in need thereof a therapeutically effective amount of a compound of formula 1 as claimed in claim 1 and/or a pharmacologically salt thereof.
